(19) United States
(12) Patent Application Publication
(10) Pub. No.: US 2007/0299129 A1
(43) Pub. Date: Dec. 27, 2007

(54) Compounds and derivatives for the treatment of medical conditions by modulating hormone-sensitive lipase activity

(75) Inventors: Jean-Frederic Sauniere, Aix-en-Provence (FR); Yat S. Or, Watertown, MA (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: DEVIRIS INC., Tortola (VG)

(21) Appl. No.: 11/650,912

(22) Filed: Jan. 5, 2007

Related U.S. Application Data
(60) Provisional application No. 60/756,376, filed on Jan. 5, 2006.
(60) Provisional application No. 60/800,338, filed on May 15, 2006.

(51) Int. Cl. A61K 31/343; A61K 31/192; A61K 31/381; A61P 3/00; A61P 3/10; C07C 69/74; C07D 317/46; C07D 333/08

(52) U.S. Cl. 514/448; 514/469; 514/568; 549/447; 549/70; 560/126

(57) ABSTRACT

The present invention discloses compounds that are inhibitors of hormone-sensitive lipase. The present invention also discloses the use of the compounds and derivatives to inhibit hormone-sensitive lipase, various pharmaceutical compositions including the compounds, and methods of treatment using these compounds and compositions.

### CROSS-REFERENCE TO RELATED APPLICATIONS

This application claims the benefit of priority to U.S. Provisional Application No. 60/800,338 filed May 15, 2006 and U.S. Provisional Application No. 60/756,376 filed Jan. 5, 2006, the disclosures of which are incorporated by reference in their entireties.

### FIELD OF THE INVENTION

The present invention relates to compounds, compositions containing them, and their use for treating medical disorders where it is desirable to decrease the activity of hormone-sensitive lipase.

### BACKGROUND OF THE INVENTION

The overall energy homeostasis of a mammalian system requires a high degree of regulation to ensure the availability of the appropriate substrate at the appropriate time. Plasma glucose levels rise during the post-prandial state, to return to pre-prandial levels within 2-3 hours. During these 2-3 hours, insulin promotes glucose uptake by skeletal muscle and adipose tissue and decreases the release of free fatty acids (FFA) from adipocytes, to ensure that the two substrates do not compete with each other. When plasma glucose levels fall, an elevation in plasma FFA is necessary to switch from glucose to fat utilization by the various tissues.

In individuals with insulin resistance, FFA levels do not fall in response to insulin, as they do in normal individuals, preventing the normal utilization of glucose by skeletal muscle, adipose and liver. Furthermore, there is a negative correlation between insulin sensitivity and plasma FFA levels.

Hormone-sensitive lipase (HSL) is an enzyme, expressed primarily in adipocytes, that catalyses the conversion of triglycerides to glycerol and fatty acids. It is through the regulation of this enzyme that the levels of circulating FFA are modulated. Insulin leads to the inactivation of HSL with a subsequent fall in plasma FFA levels during the post-prandial state, followed by the activation of the enzyme when the insulin concentration falls and catecholamines rise during the post-absorptive period. The activation of HSL leads to an increase in plasma FFA, as they become the main source of energy during fasting.

The activation-inactivation of HSL is primarily mediated through the cAMP-protein kinase A and AMP-dependent kinase pathways. There are compounds like nicotinic acid and its derivatives, which decrease the activation of HSL via these pathways and cause a decrease in lipolysis that leads to a reduction in the FFA levels. These drugs have a beneficial effect in the utilization of glucose and in the normalization of the excess triglyceride synthesis seen in patients with elevated FFA. However, because these pathways are used by other processes in the body, these drugs have severe side effects.

Thus, it is an object of the present invention to provide compounds and pharmaceutical compositions that inhibit the lipolytic activity of HSL. A further object is to provide compounds which have good pharmaceutical properties such as solubility, bioavailability, etc.

The invention relates to oleocanthals, the general structure of which is as follows, and derivatives thereof: 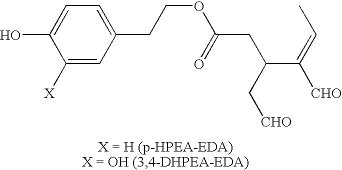
Oleocanthal (also known as deacetoxyligstroside aglycon) having the structure wherein X═H has been previously reported as having anti-COX-2 activity (Smith et al., Organic Letters, 7:5075-5078, 2005). 3,4-dihydroxyphenylelenolic acid dialdehyde (3,4-DHPEA-EDA) having the structure wherein X═OH has been previously isolated and characterized (Montedoro et al., J. Agric. Food Chem., 41: 2228-2234, 1993).

### SUMMARY OF THE INVENTION

The present invention relates to oleocanthal derivatives, analogs, and homologs of oleocanthal and related compounds, particularly prodrug derivatives of oleocanthal, analogs, and homologs, and related compounds, pharmaceutical compositions thereof, and to methods of using such derivatives and pharmaceutical compositions thereof in the treatment of disease. It has been discovered that the oleocanthals have anti-HSL activity and that the oleocanthals and their analogs and homologs and their prodrug derivatives, set forth in the compound of formulae I and II, have shown a strong inhibitory effect on the lipolytic activity of HSL and lead to a decrease in plasma FFA levels. In one aspect, the oleocanthal is para-hydroxyphenylelenolic acid dialdehyde (p-HPEA-EDA) (fraction O-4456) and 3,4-dihydroxyphenylelenolic acid dialdehyde (3,4-DHPEA-EDA) (fraction O-4457) as well as other compounds disclosed herein. These compounds can be used to treat disorders where a decreased level of plasma FFA is desired, such as insulin resistance, metabolic syndrome X, dyslipidemia, and abnormalities of lipoprotein metabolism.

The present invention is directed to the compound(s) of formula I and salts, solvates and hydrates, racemates, racemic mixtures and pure enantiomers, diastereomers, homologs, analogs and mixtures thereof,

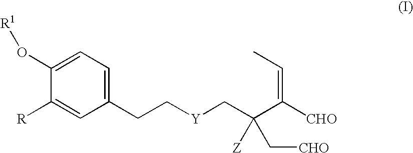

wherein R is hydrogen, hydroxyl or —OR¹;

wherein R¹ is hydrogen or a chemical moiety that can be cleaved in vivo to release a hydroxyl group and includes, for example, aliphatic or aromatic acyl (to form an ester bond) and the like. Such aliphatic or aromatic groups can include a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, or a substituted or unsubstituted heterocyclic group, a carbalkoxy, a carbaryloxy, —SO₃H and —SO₃R³, —P(O)(OH)₂, or —P(O)(OR³)₂;

wherein R³ is a saturated or unsaturated aliphatic group, substituted or unsubstituted aliphatic group, substituted or unsubstituted saturated or unsaturated alicyclic group, substituted or unsubstituted aromatic group, substituted or unsubstituted heteroaromatic group, substituted or unsubstituted heterocyclic group, glucuronide or glucuronide ester, —P(O)(OH)₂, and —P(O)(OR³)₂;

wherein Y is —(CH₂)ₘ— where m=1 or 2, —OC(O)—, —O(CH₂)ₘ− where m=1 or 2, or —S(O)ₙ(CH₂)ₘ— where m=1 or 2; n=0, 1 or 2;

wherein Z is hydrogen, CH₃, F, Cl, Br or I.

The present invention is directed to the prodrug derivative of formula I, which is set forth below in formula II, and salts, solvates and hydrates, racemates, racemic mixtures and pure enantiomers, diastereomers, homologs, analogs and mixtures thereof,

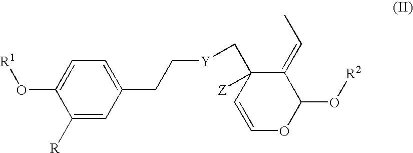

wherein R, R¹, R³, Y and Z are as previously defined; and,

wherein R² is hydrogen or a hydroxyl protecting group, as described in “Protective Groups in Organic Synthesis” by Therodora W. Greene, Peter G. M. Wuts, 1999, 3^{rd} edition, pp 17-200, —SO₃H and —SO₃R³ where R³ is a saturated or unsaturated aliphatic group; a substituted or unsubstituted saturated or unsaturated alicyclic group.

The invention also provides isolated and purified compounds of formulae (I) and (II), wherein R is hydrogen, hydroxyl or —OR¹, wherein R¹ is hydrogen, —OC(O), a carbalkoxy, a carbaryloxy, R⁴, OR⁵, SO₃H, SO₃R³, P(O)(OH)₂, P(O)(OR³)₂, glucuronide, or glucuronide ester, wherein R² is hydrogen, P, SO₃H, SO₃R³, P(O)(OH)₂, P(O)(OR³)₂, glucuronide, glucuronide ester, alkyl, substituted alkyl, alkenyl, substituted alkenyl, alkynyl, substituted alkynyl, cycloalkyl, substituted cycloalkyl, cycloalkenyl, substituted cycloalkenyl, wherein R³ is alkyl, alkenyl, alkynyl, cycloalkyl, cycloalkynl, cycloalkenyl, wherein R⁴ is hydrogen, alkyl, alkenyl, alkynyl, cycloalkyl, cycloalkynyl, aryl, heteroaryl, heterocyclylalkyl, hererocycloalkenyl, disubstituted amine, and, wherein R⁵ is any protecting group, silyl ether, C(O)R⁴.

The invention provides compounds comprising one or more of the isolated and purified compounds of formulae (I) and (II) wherein when Y is —OC(O)—, when Z is H and when R is hydrogen, R¹ is not hydrogen; and wherein when Y is —OC(O)—, when Z is H and when R is hydroxyl, R¹ is not hydrogen. The invention also provides pharmaceutical compositions comprising one or more of the isolated and purified compounds of formulae (I) and (II) wherein when Y is —OC(O)—, when Z is H and when R is hydrogen, R¹ is not hydrogen; and wherein when Y is —OC(O)—, when Z is H and when R is hydroxyl, R¹ is not hydrogen and a pharmaceutically acceptable carrier.

In one embodiment, the invention provides a method of inhibiting hormone-sensitive lipase in a patient in need thereof comprising administering to said patient one or more of the isolated and purified compounds of formulae (I) and (II), optionally in combination with one or more active ingredients which have favorable effects on metabolic disturbances or disorders, such as anti-diabetic drugs or lipid modulators.

In another embodiment, the invention provides a method of treating or preventing one or more symptoms associated with disorders of fatty acid metabolism and glucose utilization disorders in a patient in need thereof comprising administering to said patient a therapeutically effective amount of one or more of the isolated and purified compounds of formulae (I) and (II), optionally in combination with at least one further active ingredient for the treatment and/or prevention of disorders of fatty acid metabolism and glucose utilization disorders.

In another embodiment, the invention provides a method of treating or preventing disorders involving insulin resistance, such as diabetes mellitus, comprising administering to said patient a therapeutically effective amount of one or more of the isolated and purified compounds of formulae (I) and (II), optionally in combination with at least one further active ingredient for the treatment and/or prevention of disorders in which insulin resistance is involved.

In another embodiment, the invention provides a method of treating or preventing dyslipidemias and their complications comprising administering to said patient a therapeutically effective amount of one or more of the isolated and purified compounds of formulae (I) and (II).

In another embodiment, the invention provides a method of treating or preventing conditions associated with metabolic syndrome X, comprising administering to said patient a therapeutically effective amount of one or more of the isolated and purified compounds of formulae (I) and (II).

In yet another embodiment, the present invention provides pharmaceutical compositions comprising one or more of the isolated and purified compounds of formulae (I) and (II) in a pharmaceutically acceptable carrier, methods of using the isolated and purified compounds, homologs, biologically active analogs and pharmaceutical compositions thereof in the treatment of various disorders caused by an increase of hormone sensitive lipase.

### BRIEF DESCRIPTION OF THE FIGURES

FIG. 1 indicates the percentage of inhibitory activity of anti-HSL fractions (O-4456 and O-4457) in the presence of DTT.

FIG. 2 indicates the percentage of inhibitory activity of anti-HSL fractions (O-4354, O-4355 and O-4361) in the absence of DTT.

FIG. 3 indicates the percentage of inhibitory activity of anti-HSL fractions (O-4356, O-4357, O-4358, O-4359 and O-4360) in the absence of DTT.

FIG. 4 indicates the percentage of inhibitory activity of anti-HSL fractions (O-4349, O-4351, O-4352, and O-4353) in the absence of DTT.

FIG. 5 indicates the percentage of inhibitory activity of anti-HSL fractions (O-4363, O-4362, O-4364, and O-4365) in the absence of DTT.

FIG. 6 indicates the percentage of inhibitory activity of anti-HSL fractions (O-4354, O-4348, O-4366, and O-4367) in the absence of DTT.

FIG. 7 indicates the percentage of inhibitory activity of an anti-HSL fraction (O-4355) and a control (HSL+DMSO) in the absence of DTT.

FIG. 8 indicates the percentage of inhibitory activity of an anti-HSL fraction (O-4350) in the absence of DTT.

FIG. 9 indicates the percentage of inhibitory activity of an anti-HSL fraction (O-4361) in the absence of DTT.

### DETAILED DESCRIPTION OF THE PREFERRED EMBODIMENTS

Listed below are definitions of various terms used to describe this invention.

These definitions apply to the terms as they are used throughout this specification and claims, unless otherwise limited in specific instances, either individually or as part of a larger group.

An “aliphatic group” is non-aromatic moiety that may contain any combination of carbon atoms, hydrogen atoms, halogen atoms, oxygen, nitrogen or other atoms, and optionally contain one or more units of unsaturation, e.g., double and/or triple bonds. An aliphatic group may be straight chained, branched or cyclic and preferably contains between about 1 and about 24 carbon atoms, more typically between about 1 and about 12 carbon atoms. In addition to aliphatic hydrocarbon groups, aliphatic groups include, for example, alkoxyalkyls, polyalkoxyalkyls, such as polyalkylene glycols, polyamines, and polyimines, for example. Such aliphatic groups may be further substituted by one or more aliphatic substituents.

The terms “aryl” or “aromatic,” as used herein, refer to a mono- or bicyclic carbocyclic ring system having one or two aromatic rings including, but not limited to, phenyl, naphthyl, tetrahydronaphthyl, indanyl, idenyl and the like.

The terms “substituted aryl” or “substituted aromatic” as used herein, refer to an aryl group, as previously defined, substituted by one, two, three or more aromatic substituents.

The terms “heteroaryl or “heteroaromatic,” as used herein, refer to a mono-, bi-, or tri-cyclic aromatic radical or ring having from five to ten ring atoms of which at least one ring atom is selected from S, O and N; zero, one, two, three or more ring atoms are additional heteroatoms independently selected from S, O and N; and the remaining ring atoms are carbon, wherein any N or S contained within the ring may be optionally oxidized. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, tetrazolyl and the like. The heteroaromatic ring may be bonded to the chemical structure through a carbon or hetero atom.

The terms “substituted heteroaryl” or “substituted heteroaromatic,” as used herein, refer to a heteroaryl group as previously defined, substituted by one, two, three or more aromatic substituents.

The term “alicyclic,” as used herein, denotes a monovalent group derived from a monocyclic or bicyclic saturated carbocyclic ring compound by the removal of a single hydrogen atom. Examples include, but not limited to, cyclopropyl, cyclobutyl, cyclopentyl, cyclohexyl, bicyclo[2.2.1]heptyl, and bicyclo[2.2.2]octyl.

The term “substituted alicyclic” group as previously defined, substituted by one, two, three or more aliphatic substituents.

The terms “heterocyclic” as used herein, refers to a non-aromatic 5-, 6- or 7-membered ring or a bi- or tri-cyclic group fused system, where (i) each ring contains between one and three heteroatoms independently selected from oxygen, sulfur and nitrogen, (ii) each 5-membered ring has 0 to 1 double bonds and each 6-membered ring has 0 to 2 double bonds, (iii) the nitrogen and sulfur heteroatoms may optionally be oxidized, (iv) the nitrogen heteroatom may optionally be quaternized, (iv) any of the above rings may be fused to a benzene ring, and (v) the remaining ring atoms are carbon atoms which may be optionally oxo-substituted. Representative heterocycloalkyl groups include, but are not limited to, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, tetrahydrofuryl, and the like.

The term “carbalkyloxy,” as used herein refers to —C(═O)OR, wherein R is an alkyl group.

The term “carbaryloxy,” as used herein refers to —C(═O)OR, wherein R is an aryl group.

The term “substituted heterocyclic,” as used herein, refers to a heterocyclic group, as previously defined, substituted by one, two, three or more aliphatic substituents.

Suitable aliphatic or aromatic substituents include, but are not limited to, —F, —Cl, —Br, —I, —OH, protected hydroxy, aliphatic ethers, aromatic ethers, oxo, —NO₂, —CN, —C₁-C₁₂-alkyl optionally substituted with halogen (such as perhaloalkyls), C₂-C₁₂-alkenyl optionally substituted with halogen, —C₂-C₁₂-alkynyl optionally substituted with halogen, —NH₂, protected amino, —NH—C₁-C₁₂-alkyl, —NH—C₂-C₁₂-alkenyl, —NH—C₂-C₁₂-alkynyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₁₂-alkenyl, —O—C₂-C₁₂-alkynyl, —O—C₃-C₂-cycloalkyl, —O-aryl, —O-heteroaryl, —O-heterocycloalkyl, —C(O)—C₁-C₁₂-alkyl, —C(O)—C₂-C₁₂-alkenyl, —C(O)—C₂-C₁₂-alkynyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₁₂-alkenyl, —CONH—C₂-C₁₂-alkynyl, —CONH—C₃-C₁₂-cycloalkyl, —CONH-aryl, —CONH-heteroaryl, —CONH-heterocycloalkyl, —CO₂—C₁-C₁₂-alkyl, —CO₂—C₂-C₁₂-alkenyl, —CO₂—C₂-C₁₂-alkynyl, —CO₂—C₃-C₁₂-cycloalkyl, —CO₂-aryl, —CO₂-heteroaryl, —CO₂-heterocycloalkyl, —OCO₂—C₁-C₁₂-alkyl, —OCO₂—C₂-C₁₂-alkenyl, —OCO₂—C₂-C₁₂-alkynyl, —OCO₂—C₃-C₁₂-cycloalkyl, —OCO₂-aryl, —OCO₂-heteroaryl, —OCO₂-heterocycloalkyl, —OCONH₂, —OCONH—C₁-C₁₂-alkyl, —OCONH—C₂-C₁₂-alkenyl, —OCONH—C₂-C₁₂-alkynyl, —OCONH—C₃-C₁₂-cycloalkyl, —OCONH-aryl, —OCONH-heteroaryl, —OCONH-heterocycloalkyl, —NHC(O)—C₁-C₁₂-alkyl, —NHC(O)—C₂-C₁₂-alkenyl, —NHC(O)—C₂-C₁₂-alkynyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₁₂-alkenyl, —NHCO₂—C₂-C₁₂-alkynyl, —NHCO₂—C₃-C₁₂-cycloalkyl, —NHCO₂-aryl, —NHCO₂— heteroaryl, —NHCO₂-heterocycloalkyl, —NHC(O)NH₂, NHC(O)NH—C₁-C₁₂-alkyl, —NHC(O)NH—C₂-C₁₂-alkenyl, —NHC(O)NH—C₂-C₁₂-alkynyl, —NHC(O)NH—C₃-C₁₂-cycloalkyl, —NHC(O)NH-aryl, —NHC(O)NH-heteroaryl, —NHC(O)NH-heterocycloalkyl, NHC(S)NH₂, NHC(S)NH—C₂-C₁₂-alkyl, —NHC(S)NH—C₂-C₁₂-alkenyl, —NHC(S)NH—C₂-C₁₂-alkynyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, NHC(NH)NH—C₁-C₁₂-alkyl, —NHC(NH)NH—C₂-C₁₂-alkenyl, —NHC(H)NH—C₂-C₁₂-alkynyl, —NHC(O)NH—C₃-C₁₂-cycloalkyl, —NHC(NH)NH-aryl, —NHC(NH)NH-heteroaryl, —NHC(NH)NH-heterocycloalkyl, NHC(NH)—C₁-C₁₂-alkyl, —NHC(NH)—C₂-C₁₂-alkenyl, —NHC(NH)—C₂-C₁₂-alkynyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₁₂-alkenyl, —C(NH)NH—C₂-C₁₂-alkynyl, —C(NH)NH—C₃-C₁₂-cycloalkyl, —C(NH)NH-aryl, —C(NH)NH-heteroaryl, —C(NH)NH-heterocycloalkyl, —S(O)—C₁-C₁₂-alkyl, —S(O)—C₂-C₁₂-alkenyl, —S(O)—C₂-C₁₂-alkynyl, —S(O)—C₃-C₁₂-cycloalkyl, —S(O)-aryl, —S(O)-heteroaryl, —S(O)-heterocycloalkyl —SO₂NH₂, —SO₂NH—C₁-C₁₂-alkyl, —SO₂NH—C₂-C₁₂-alkenyl, —SO₂NH—C₂-C₁₂-alkynyl, —SO₂NH—C₃-C₁₂-cycloalkyl, —SO₂NH-aryl, —SO₂NH— heteroaryl, —SO₂NH— heterocycloalkyl, —NHSO₂—C₁-C₁₂-alkyl, —NHSO₂—C₂-C₁₂-alkenyl, —NHSO₂—C₂-C₁₂-alkynyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₁₂-alkenyl, —S—C₂-C₁₂-alkynyl, —S—C₃-C₁₂-cycloalkyl, —S-aryl, —S-heteroaryl, —S-heterocycloalkyl, or methylthiomethyl. It is understood that the aryls, heteroaryls, alkyls and the like can be further substituted.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid or inorganic acid. Examples of pharmaceutically acceptable nontoxic acid addition salts include, but are not limited to, salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid lactobionic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention. “Prodrug”, as used herein means a compound which is convertible in vivo by metabolic means (e.g. by hydrolysis) to a compound of Formula I. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed).

“Design and Application of Prodrugs, Textbook of Drug Design and Development, Chapter 5, 113-191 (1991); Bundgaard, et al., Journal of Drug Deliver Reviews, 8:1-38 (1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

As used herein, “pharmaceutically acceptable carrier” is intended to include any and all solvents, dispersion media, coatings, antibacterial and antifungal agents, isotonic and absorption delaying agents, and the like, compatible with pharmaceutical administration, such as sterile pyrogen-free water. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The present invention relates to the discovery of the hormone sensitive lipase (HSL) strong inhibitory activity of the compounds set forth in formulae I and II and their derivatives, particularly prodrug derivatives, pharmaceutical compositions thereof, and to methods of using such derivatives, homologs biologically active analogs and pharmaceutical compositions thereof in the treatment of disease.

The chemical structure of oleocanthal is as follows:

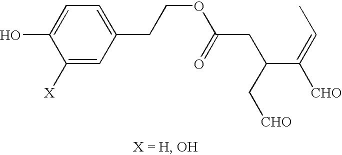

These compounds may be extracted from olives, olive oil, roots, bark and leaves from olea europeae species (Paiva and al., J. Agric. Food Chem., 49:4214-4219, 2001). The compounds of the invention may possess pharmaceutical and chemical properties which render them superior over the natural oleocanthal, and its analogs and metabolites, as therapeutic agents.

As described above, the present invention relates to the discovery of anti-HSL activity of the compounds of the formulae (I) and (II) 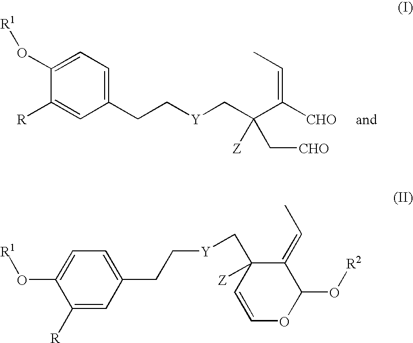
described in the form of their salts, solvates and hydrates, racemates, racemic mixtures and pure enantiomers, and to their diastereomers, homologs, analogs and mixtures thereof, and,

wherein R is hydrogen, hydroxyl or —OR¹ wherein R¹ is hydrogen or a chemical moiety that can be cleaved in vivo to release a hydroxyl group and includes, for example, aliphatic or aromatic acyl (to form an ester bond) and the like. Such aliphatic or aromatic groups can include a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, or a substituted or unsubstituted heterocyclic group. In addition, R¹ is —SO₃H and —SO₃R³ where R³ is a saturated or unsaturated aliphatic group; a substituted or unsubstituted saturated or unsaturated alicyclic group; —P(O)(OH)₂, and —P(O)(OR³)₂ where R³ is as previously defined; glucuronide and esters of glucuronide, R is hydrogen or a hydroxyl protecting group, as described in “Protective Groups in Organic Synthesis” by Therodora W. Greene, Peter G. M. Wuts, 1999, 3rd edition, pp 17-200. In addition, R² is —SO₃H and —SO₃R³ where R³ is a saturated or unsaturated aliphatic group; a substituted or unsubstituted saturated or unsaturated alicyclic group; —P(O)(OH)₂, and —P(O)(OR³)₂ where R³ is as previously defined; glucoronide and esters of glucoronide,

wherein Y is —(CH₂)ₘ— where m=1 or 2, —OC(O)—, —O(CH₂)ₘ— where m=1 or 2, or —S(O)ₙ(CH₂)ₘ— where m=1 or 2; n=0, 1 or 2;

wherein Z is hydrogen, CH₃, F, Cl, Br or I.

In a preferred embodiment, R=hydrogen or —OR¹; R¹ is —COR⁴ or —C(═O)—O—R⁴. R⁴ is independently hydrogen, a di-substituted amino, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, or a substituted or unsubstituted heterocyclic group. In a preferred embodiment, R⁴ is methyl, ethyl, propyl, isobutyl, benzyl, methylbenzyl, substituted or unsubstituted aryl; substituted or unsubstituted heterocyclic. R² is substituted or unsubstituted saturated or unsaturated aliphatic group; substituted or unsubstituted alicylic group. In addition, R² is —SO₃H and —SO₃R³ where R³ is a saturated or unsaturated aliphatic group; a substituted or unsubstituted saturated or unsaturated alicyclic group; —P(O)(OH)₂, and —P(O)(OR³)₂ where R³ is as previously defined; glucuronide and glucuronide esters.

The compounds of the invention may possess pharmaceutical and chemical properties which render them superior over the natural oleocanthal, analogs and its metabolites as therapeutics. This invention, in addition to the compound of formulae (I) and (II), also contemplates the use of homologs and analogs of such compounds. In this context, homologs are molecules having substantial structural similarities to the above-described compounds and analogs are molecules having substantial biological similarities regardless of structural similarities.

The invention also provides methods for treating conditions where a decrease and inhibition of hormone sensitive lipase (HSL) is beneficial to patients to treat disorders where a decreased level of plasma FFA is desired, such as insulin resistance, metabolic syndrome X, dyslipidemia, and abnormalities of lipoprotein metabolism.

The compounds of the invention of the general formulae I or II may have a strong inhibitory effect on HSL, an allosteric enzyme in adipocytes which is inhibited by insulin and is responsible for the breakdown of fats in fat cells and thus for transferring fat constituents into the blood stream. Inhibition of this enzyme is therefore equivalent to an insulin-like effect of the compounds and derivatives of the invention, eventually leading to reduction of free fatty acids in the blood and of blood glucose. The compounds of formulae I or II can therefore be employed for metabolic derangements such as, for example, for non-insulin-dependent diabetes mellitus, for diabetic syndrome and for direct pancreatic damage.

Synthetic Schemes

The compounds and processes of the present invention will be better understood in connection with synthetic schemes that illustrate the methods by which the compounds of the invention may be prepared. As shown in general scheme 1, oleocanthal, obtained from virgin olive oil extract or prepared according to the procedures described in literature (AB Smith et al, Organic Letters, Vol 7, No 22, 5075-5078, 2005) is treated with an acid anhydride in the presence of a base and in an inert solvent, in a temperature ranging from −70° C. to 170° C. Examples of appropriate bases included, but are not limited to, pyridine, triethyl amine, Hunig's base, dimethylaminopyridine, CsHCO₃, KHCO₃, or K₂CO₃. Examples of appropriate inert solvents include, but are not limited to, chloroform, methylene chloride, tetrahydrofuran, diethylether, benzene, toluene, xylene, DMF, and N-methylpyrolidone etc. The product is obtained by an aqueous work up and extracted to organic solvent and purified by chromatography, if necessary.

The products of Scheme 1 can also be prepared according to the procedures described in “Protective Groups in Organic Synthesis” by Therodora W. Greene, Peter G. M. Wuts, 1999, 3rd edition.

### Scheme-1

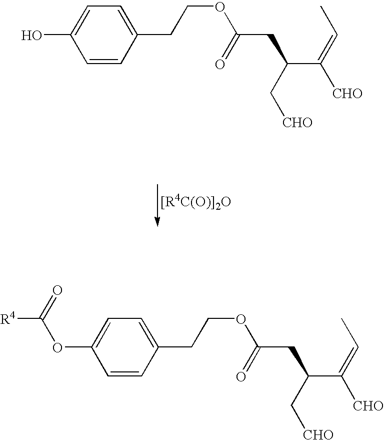

As shown in scheme 2, examples of appropriate base include, but are not limited to, pyridine, triethyl amine, Hunig's base, dimethylaminopyridine, CsHCO₃, KHCO₃, or K₂CO₃. Examples of solvents are, but not limited to, chloroform, methylene chloride, tetrahydrofuran, diethylether, benzene, toluene, xylene, DMF, and N-methylpyrolidone, etc. The product is obtained by an aqueous work up and extracted to organic solvent and purified by chromatography, if necessary.

The products of Scheme 2 can also be prepared according to the procedures described in “Protective Groups in Organic Synthesis” by Therodora W. Greene, Peter G. M. Wuts, 1999, 3rd edition.

### Scheme 2

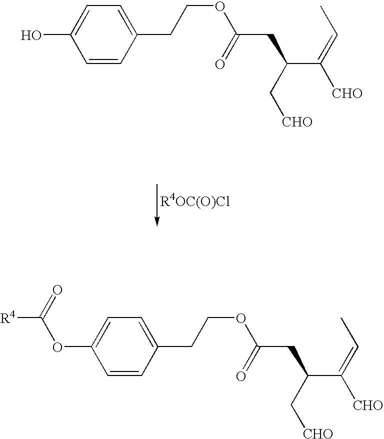

Scheme 3 shows the formation of enol-lactol by reacting oleocanthal or protected oleocanthal with acid or base in an appropriate solvent followed by aqueous work up. Examples of acids include, but are not limited to, acetic acid, formic acid, lactobionic acid, malic acid, toluensulfonic acid, methansulfonic acid, dilute sulfuric acid, dilute phosphoric acid, etc. Examples of bases included, but are not limited to, pyridine, dimethylaminopyridine, methylated guanidine, Hunig's base, etc. Appropriate solvents include, but are not limited to, tetrahydrofuran, diethyl ether, glyme, diglyme, water, DMF, DMSO, etc.

### Scheme 3

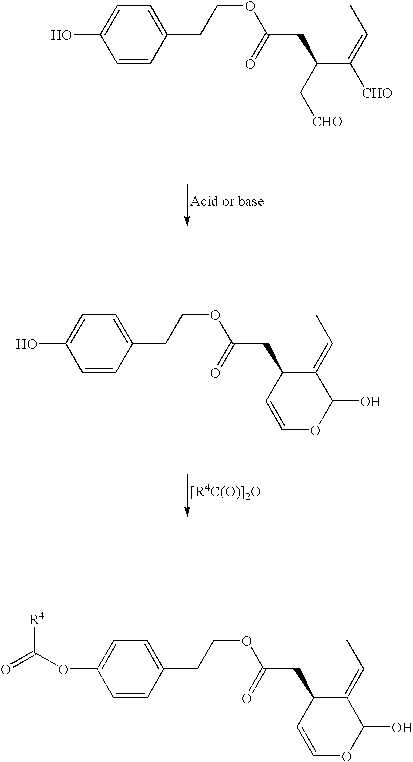

In Scheme 4, activation is achieved by acids, BF₃, ionic resins, zeolites, molecular sieves, acyl chloride, sulfonic anhydride or triflorosulfonyl anhydride, etc., the corresponding alcohol can be used as a solvent or in the presence of second solvent as previously described. Experimental procedures are exemplified, but not limited to, the following literature publications: Reissig, H.-U.; Tetrahedron Lett 1981, 22, 2981; Leroux et al., Carbohydr Res 1978, 67, 163; Rachaman et al., Carbohydr Res 1978, 67, 147; Johnson et al., J Am Chem Soc 1962, 84, 989; Smith et al., J Am Chem Soc 1955, 77, 3159; Osman et al., J Am Chem Soc 1951, 73, 2726; Cadotte et al., J Am Chem Soc 1952, 74, 1501; Mulard et al., Carbohydr Res 1994, 259 (1), 21-34; Petit, et al., Synthesis 1995, (12), 1517-1520; Bleriot, et al. Tetrahedron: Asymmetry 1996, 7 (9), 2761-2772; and Li et al.; Tetrahedron 2001, 57 (20), 4297-4309.

### Scheme 4

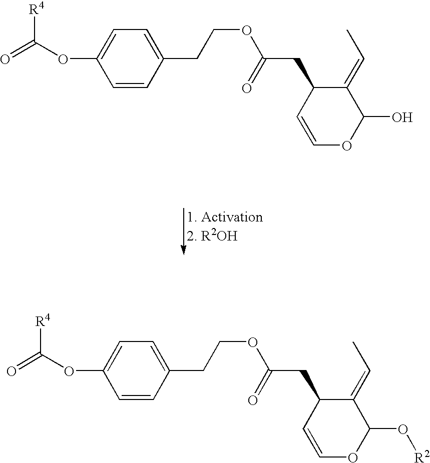

In Scheme 5, examples of appropriate base include, but are not limited to, pyridine, triethyl amine, Hunig's base, dimethylaminopyridine, CsHCO₃, KHCO₃, or K₂CO₃. Examples of appropriate solvents include, but are not limited to, chloroform, methylene chloride, tetrahydrofuran, diethylether, benzene, toluene, xylene, DMF, and N-methylpyrolidone, etc. The product is obtained by an aqueous work up and extracted to organic solvent and purified by chromatography if necessary. MODRO, A. M.; MODRO, T. A.; Org Prep Proced Int 1992, 24 (1), 57-60.

### Scheme 5

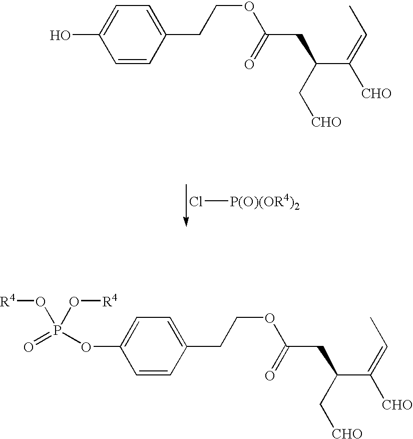

As shown in general Scheme 6, compound (1-1) was prepared according to the literature, AB Smith et al, Organic Letters, Vol 7, No 22, 5075-5078, 2005. By reacting compound (1-1) with strong base such as HMPA or tert-Butyl Lithium in appropriate solvent, followed by alkylation with corresponding amide provides compound (1-2) according to the procedures described in the above reference. In compound (1-2), Y is as previously described. Compound (1-2) is elaborated to the target compound according to the procedures described in the literature.

### Scheme 6

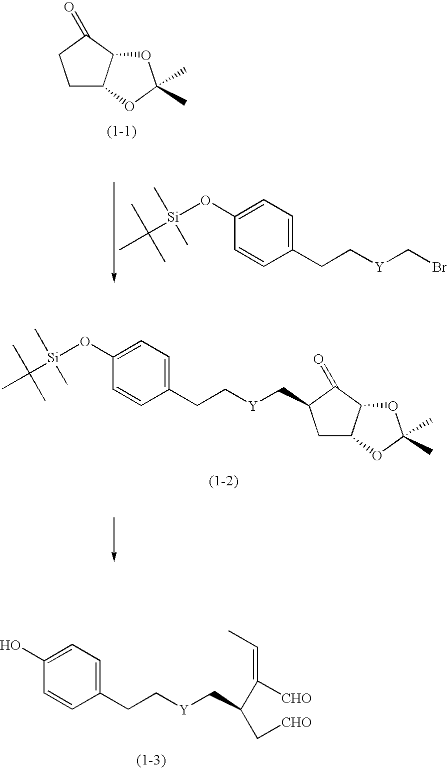

As shown in Scheme 7, compound (2-1) was prepared by generating alpha-anion with strong base and trapped with electrophile Z. Examples of electrophilic reagents are methyl iodide, N-bromosuccinamide, fluorinating agents and iodine. Alkylation followed by elaborations produced the targeted compound (2-3). The procedure from the previous reference is used to prepare the compounds described in Scheme-7. In scheme-7, X, Y and Z are as previously defined. Examples of Z-W include, but are not limited to, CH₃—I, N-bromosuccinamide, ArC(O)NR—F, and I₂.

### Scheme 7

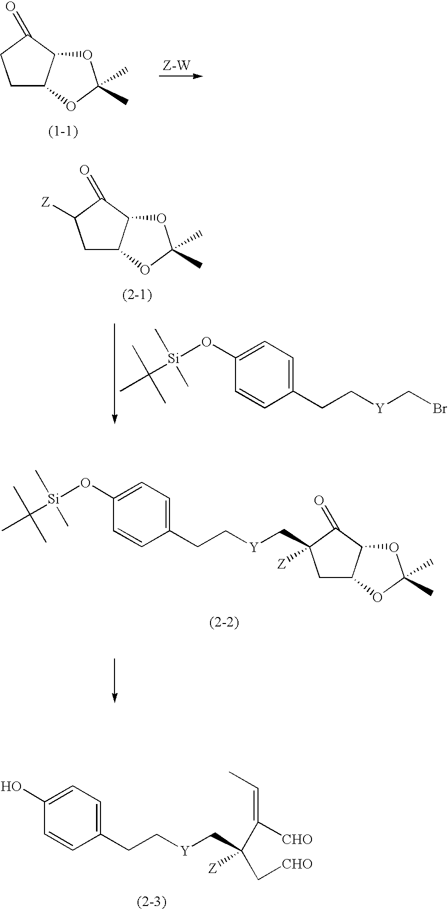 Therapeutic Uses

The compounds of the invention of the general formulae (I) and (II) have a strong inhibitory effect on hormone sensitive lipase (HSL), an allosteric enzyme in adipocytes which is inhibited by insulin and is responsible for the breakdown of fats in fat cells and thus for transferring fat constituents into the blood stream. Inhibition of this enzyme is therefore equivalent to an insulin-like effect of the compounds and derivatives of the invention, eventually leading to reduction of free fatty acids in the blood and of blood glucose. These compounds can therefore be employed for metabolic derangements such as, for example, for non-insulin-dependent diabetes mellitus, for diabetic syndrome and for direct pancreatic damage.

Compounds of formulae (I) and (II) are particularly suitable for the treatment and/or prevention of alterations of fatty acid metabolism and glucose utilization disorders, disorders in which insulin resistance is involved, e.g., Diabetes mellitus, especially type-II diabetes, including the prevention of the complications associated therewith. Particular aspects in this connection are hyperglycemia, improvement in insulin resistance, improvement in glucose tolerance, protection of the pancreatic β-cells, and prevention of macro- and microvascular disorders. Compounds of formulae (I) and (II) are also suitable for the treatment and/or prevention of dyslipidemias their complications such as, for example, atherosclerosis, coronary heart disease, cerebrovascular disorders, etc, especially those (but not restricted thereto) which are characterized by one or more of the following factors: high plasma triglyceride concentrations, high postprandial plasma triglyceride concentrations, low HDL cholesterol concentration, low ApoA lipoprotein concentrations, high LDL cholesterol concentrations, small dense LDL cholesterol particles and high ApoB lipoprotein concentrations.

Various other conditions may be associated with the metabolic syndrome X, such as: obesity, including central obesity, thromboses, heart failure such as, for example (but not restricted thereto), following myocardial infarction, hypertensive heart disease or cardiomyopathy.

In still another aspect, one or more of the compounds of general formulae (I) and (II) are useful for the treatment of hyperglycemia, elevated HbAlc level, hyperinsulinemia, type II diabetes, latent autoimmune diabetes in adults, maturity onset diabetes, beta-cell apoptosis, hemochromatosis induced diabetes, impaired glucose tolerance, impaired fasting glucose, metabolic syndrome X, insulin resistance, impaired lipid tolerance, cystic fibrosis-related diabetes, polycystic ovarian syndrome, and gestational diabetes.

In still another aspect, one or more of the compounds of general formulae (I) and (II) may be useful for the treatment of liver disorders, such as hepatic steatosis and cirrhosis.

In still another aspect, one or more of the compounds of general formulae (I) and (II) may be useful for the treatment of symptoms such as weight loss and cachexia associated with AIDS or an AIDS related diseases. Also, conditions or disorders, such as osteoarthritis; lupus erythematosus (LE) or inflammatory rheumatic disorders such as, for example, rheumatoid arthritis; vasculitis; wasting (cachexia); gout; ischemia/reperfusion syndrome acute respiratory distress syndrome (ARDS); lipodystrophy and lipodystrophic states, also for treating adverse effects of other drugs used to treat various conditions (e.g. following medicaments for treating HIV or tumors).

In still another aspect, one or more of the compounds of general formulae (I) and (II) are useful for the prevention or treatment of obesity, dyslipidemia, diabetic dyslipidemia, hyperlipidemia, hypertriglyceridemia, hyperlipoproteinemia, hypercholesterolemia, hypertension, essential hypertension, acute hypertensive emergency, arteriosclerosis, atherosclerosis, restenosis, intermittent claudication, cardiovascular disease, cardiomyopathy, cardiac hypertrophy, left ventricular hypertrophy, coronary artery disease, early coronary artery disease, heart insufficiency, exercise tolerance, chronic heart failure, mild chronic heart failure, arrhythmia, cardiac dysrythmia, syncopia, heart attack, myocardial infarction, Q-wave myocardial infarction, stroke, acute coronary syndrome, angina pectoris, unstable angina, cardiac bypass reocclusion, diastolic dysfunction, systolic dysfunction, non-Q-wave cardiac necrosis, catabolic changes after surgery, acute pancreatitis, and irritable bowel syndrome.

In still another aspect, one or more of the compounds of general formulae (I) and (II) may be useful for the prevention or treatment of diabetic retinopathy, background retinopathy, preproliferative retinopathy, proliferative retinopathy, macular edema, cataracts, nephropathy, nephrotic syndrome, diabetic nephropathy, microalbuminuria, macroalbuminuria, neuropathy, diabetic neuropathy, polyneuropathy, and diabetic autonomic neuropathy.

In still another aspect, one or more of the compounds of general formulae (I) and (II) may be useful for the prevention or treatment of other disorders or conditions in which inflammatory reactions or cell differentiation may be involved. For example, atherosclerosis such as, for example (but not restricted thereto), coronary sclerosis including angina pectoris or myocardial infarction, stroke vascular restenosis or reocclusion; chronic inflammatory bowel diseases such as, for example, Crohn's disease and ulcerative colitis, pancreatitis; and other inflammatory states.

In still another aspect, one or more of the compounds of general formulae (I) and (II) are believed to be useful for the prevention or treatment of a disease, condition or disorder wherein cholesterol is a precursor. Such diseases, conditions or disorders may relate to testosterone, e.g. male contraception, excessive testosterone levels, and prostate cancer. They may also relate to cortisol or corticotropin, e.g. Cushing disease.

The compounds of the invention are also believed to be useful for the prevention or treatment of cancer. Thus, one or more of the compounds of general formulae (I) and (II) may be useful for the treatment of insulinoma (pancreatic islet cell tumors), e.g. malignant insulinomas and multiple insulinomas, adipose cell carcinomas, e.g. lipocarcinoma adipose cell tumors; pomatous carcinomas such as, for example, liposarcomas; solid tumors and neoplasms such as, for example (but not restricted thereto), carcinomas of the gastrointestinal tract, liver, biliary tract and pancreas; endocrine tumors; carcinomas of the lungs, kidneys, urinary tract, genital tract, and prostate.

The compounds of the invention are also believed to be useful for the prevention or treatment of phaechromocytoma and other diseases with increased catecholamine incretion.

The compounds of the invention are also believed to be useful for the prevention or treatment of prostate cancer, e.g. adenocarcinoma, acute and chronic myeloproliferative disorders and lymphomas; angiogenesis, cancer associated cachexia; neurodegenerative disorders, such as Alzheimer's disease, multiple sclerosis, and Parkinson's disease; erythemato-squamous dermatoses such as, psoriasis, acne vulgaris; and other skin disorders and dermatological conditions which are modulated by PPAR, including but not limited to, eczemas and neurodermatitis; dermatitis such as, for example, seborrheic dermatitis or photodermatiti; keratitis and keratoses such as, for example, seborrheic keratoses, senile keratoses, actinic keratosis, photo-induced keratoses or keratosis follicularis, keloids and keloid prophylaxis; warts, including condylomata or condylomata acuminata; human papilloma viral (HPV) infections such as, for example, venereal papillomata, viral warts such as, for example, molluscum contagiosum, leukoplakia, papular dermatoses such as, for example, lichen planus; skin cancer such as, for example, basal-cell carcinomas, melanomas or cutaneous T-cell lymphomas, localized benign epidermal tumors such as, for example, keratoderma, epidermal naevi, high blood pressure, metabolic syndrome X; polycystic ovary syndrome (PCOS); and asthma.

Combination Therapy

The compounds of the invention can be administered alone or in combination with one or more further pharmacologically active substances which have, for example, favorable effects on metabolic disturbances or disorders frequently associated therewith. Examples of such medicaments are medicaments which lower blood glucose, anti-diabetics, active ingredients for the treatment of dyslipidemias, anti-atherosclerotic medicaments, anti-obesity agents, anti-inflammatory active ingredients, active ingredients for the treatment of malignant tumors, anti-thrombotic active ingredients, active ingredients for the treatment of high blood pressure, active ingredients for the treatment of heart failure and active ingredients for the treatment and/or prevention of complications caused by diabetes or associated with diabetes.

Furthermore, the present compounds may be administered in combination with one or more anti-hypertensive agents. Examples of antihypertensive agents are β-blockers such as alprenolol, atenolol, timolol, pindolol, propranolol and metoprolol, ACE (angiotensin converting enzyme) inhibitors such as benazepril, captopril, alatriopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin. Any suitable combination of the compounds according to the invention with one or more of the above-mentioned compounds and optionally one or more further pharmacologically active substances are considered to be within the scope of the present invention.

The one or more further pharmacologically active substances can be combined with one or more of the compounds of general formulae (I) and (II) in particular for a synergistic improvement in the effect. Administration of the active ingredient combination can take place either by separate administration of the active ingredients to the patient or in the form of combination products.

In one embodiment of the invention, one or more of the compounds of general formulae (I) and (II) are administered in combination with an anti-diabetic (see, e.g., Rote Liste 2001, chapter 12 or in the USP Dictionary of USAN and International Drug Names, US Pharmacopeia, Rockville 2001). Antidiabetics include all insulins and insulin derivatives and other fast-acting insulins, GLP-1 receptor modulators.

The orally effective hypoglycemic active ingredients may include but are not limited to, sulfonylureas (such as tolbutamide, glibenclamide, glipizide or glimepiride), biguanides (such as metformin), meglitinides (such as repaglinide), oxadiazolidinediones, thiazolidinediones (such as ciglitazone, pioglitazone, rosiglitazone), glucosidase inhibitors, glucagon antagonists, GLP-1 agonists, DPP-IV inhibitors, potassium channel openers, insulin sensitizers, inhibitors of liver enzymes involved in the stimulation of gluconeogenesis and/or glycogenolysis, modulators of glucose uptake, compounds which alter lipid metabolism and lead to a change in the blood lipid composition, compounds which reduce food intake, PPAR and PXR modulators and active ingredients which act on the ATP-dependent potassium channel of the beta cells.

In one embodiment of the invention, one or more of the compounds of general formulae (I) and (II) are administered in combination with a PPARgamma agonist such as, for example, rosiglitazone, pioglitazone.

In one embodiment, one or more of the compounds of general formulae (I) and (II) are administered in combination with an α-glucosidase inhibitor such as, for example, miglitol or acarbose.

In one embodiment, one or more of the compounds of general formulae (I) and (II) are administered in combination with more than one of the aforementioned compounds, e.g. in combination with a sulfonylurea and metformin, a sulfonylurea and acarbose, repaglinide and metformin, insulin and a sulfonylurea, insulin and metformin, insulin and troglitazone, insulin and lovastatin, etc.

In one embodiment of the invention, one or more of the compounds of general formulae (I) and (II) are administered in combination with one or more lipid modulators. Exemplary lipid modulators include, but are not limited to, HMGCoA reductase inhibitor (such as lovastatin, fluvastatin, pravastatin, simvastatin, ivastatin, itavastatin, atorvastatin, rosuvastatin); bile acid reabsorption inhibitors; polymeric bile acid adsorbent (such as, cholestyramine, colesevelam); cholesterol absorption inhibitor (such as ezetimibe, tiqueside, pamaqueside); an LDL receptor inducer;

In one embodiment, one or more of the compounds of general formulae (I) and (II) are administered in combination with bulking agents.

In one embodiment of the invention, one or more of the compounds of general formulae (I) and (II) are administered in combination with a PPARalpha agonist.

In one embodiment of the invention, one or more of the compounds of general formulae (I) and (II) are administered in combination with a mixed PPAR alpha/gamma agonist such as, for example, AZ 242, Tesaglitazar.

In one embodiment of the invention, one or more of the compounds of general formulae (I) and (II) are administered in combination with a fibrate such as, for example, fenofibrate, gemfibrozil, clofibrate, bezafibrate.

In one embodiment of the invention, one or more of the compounds of general formulae (I) and (II) are administered in combination with nicotinic acid or niacin.

In one embodiment of the invention, one or more of the compounds of general formulae (I) and (II) are administered in combination with a CETP inhibitor alone such as, e.g. CP-529, 414 (torcetrapib) and in a multiple combination therapy including but not restricted to HMGCoA reductase inhibitor such as lovastatin, fluvastatin, pravastatin, simvastatin, ivastatin, atorvastatin, rosuvastatin.

In one embodiment of the invention, one or more of the compounds of general formulae (I) and (II) are administered in combination with an ACAT inhibitor.

In one embodiment of the invention, one or more of the compounds of general formulae (I) and (II) are administered in combination with an MTP inhibitor such as, for example, implitapide.

In one embodiment of the invention, one or more of the compounds of general formulae (I) and (II) are administered in combination with an antioxidant.

In one embodiment of the invention, one or more of the compounds of general formulae (I) and (II) are administered in combination with a lipoprotein lipase inhibitor.

In one embodiment of the invention, one or more of the compounds of general formulae (I) and (II) are administered in combination with an ATP citrate lyase inhibitor.

In one embodiment of the invention, one or more of the compounds of general formulae (I) and (II) are administered in combination with a squalene synthetase inhibitor.

In one embodiment of the invention, one or more of the compounds of general formulae (I) and (II) are administered in combination with a lipoprotein(s) antagonist.

In another embodiment of the invention, one or more of the compounds of general formulae (I) and (II) are administered in combination with an anti-obesity agent. In one embodiment of the invention, the compounds of the formula (I) or (II) are administered in combination with a lipase inhibitor such as, for example, orlistat.

In one embodiment, the further active ingredient is fenfluramine, dexfenfluramin or sibutramine.

In a further embodiment, one or more of the compounds of general formulae (I) and (II) are administered in combination with CART modulators, NPY antagonists, MC4 agonists, orexin antagonists, H3 agonists, TNF agonists, CRF antagonists, CRF BP antagonists, urocortin agonists, β3 agonists, MSH (melanocyte-stimulating hormone) agonists, CCK-A agonists, serotonin reuptake inhibitors (e.g. dexfenfluramine), mixed serotoninergic and noradrenergic compounds, 5HT agonists, bombesin agonists, galanin antagonists, growth hormone (e.g. human growth hormone), growth hormone-releasing compounds, TRH agonists, uncoupling protein 2 or 3 modulators, leptin agonists, DA agonists (bromocriptine, Doprexin), lipase/amylase inhibitors, PPAR modulators, RXR modulators or TR-β agonists.

In one embodiment of the invention, the further active ingredient is leptin, dexamphetamine, amphetamine, mazindole or phentermine.

In one embodiment, one or more of the compounds of general formulae (I) and (II) are administered in combination with medicaments having effects on the coronary circulation and the vascular system, such as, for example, ACE inhibitors (e.g. ramipril), medicaments which act on the angiotensin-renine system, calcium antagonists, beta blockers etc.

In one embodiment, one or more of the compounds of general formulae (I) and (II) are administered in combination with medicaments having an anti-inflammatory effect.

In one embodiment, one or more of the compounds of general formulae (I) and (II) are administered in combination with medicaments which are employed for cancer therapy and cancer prevention.

It will be appreciated that every suitable combination of the compounds of the invention with one or more of the aforementioned compounds and optionally one or more other pharmacologically active substances is regarded as falling within the protection conferred by the present invention.

Pharmaceutical Compositions

The invention encompasses pharmaceutical compositions comprising pharmaceutically acceptable salts of the compounds, or derivatives, analogs, homologs thereof, of the invention as described above. The invention also encompasses pharmaceutical compositions comprising hydrates of the compounds of the invention. The term “hydrate” includes but is not limited to hemihydrate, monohydrate, dehydrate, trihydrate and the like. The invention further encompasses pharmaceutical compositions comprising any solid or liquid physical form of the compound of the invention. For example, the compounds can be in a crystalline form, in amorphous form, and have any particle size. The particles may be micronized, or may be agglomerated, particulate granules, powders, oils, oily suspensions or any other form of solid or liquid physical form.

The compounds of the invention, and derivatives, fragments, analogs, homologs, pharmaceutically acceptable salts or hydrate thereof can be incorporated into pharmaceutical compositions suitable for administration, together with a pharmaceutically acceptable carrier or excipient. Such compositions typically comprise a therapeutically effective amount of any of the compounds above, and a pharmaceutically acceptable carrier. Preferably, the effective amount when treating cancer is an amount effective to selectively induce terminal differentiation of suitable neoplastic cells and less than an amount which causes toxicity in a patient.

Prodrugs described in formula (I) and (II) may be administered by any suitable means, including, without limitation, parenteral, intravenous, intramuscular, subcutaneous, implantation, oral, sublingual, buccal, nasal, pulmonary, transdermal, topical, vaginal, rectal, and transmucosal administrations or the like. Pharmaceutical preparations include a solid, semisolid or liquid preparation (tablet, pellet, troche, capsule, suppository, cream, ointment, aerosol, powder, liquid, emulsion, suspension, syrup, injection etc.) containing an oleocanthal as an active ingredient, which is suitable for selected mode of administration. In one embodiment, the pharmaceutical compositions are administered orally, and are thus formulated in a form suitable for oral administration, i.e., as a solid or a liquid preparation. Suitable solid oral formulations include tablets, capsules, pills, granules, pellets, sachets and effervescent, powders, and the like. Suitable liquid oral formulations include solutions, suspensions, dispersions, emulsions, oils and the like. In one embodiment of the present invention, the composition is formulated in a capsule. In accordance with this embodiment, the compositions of the present invention comprise in addition to the active compound and the inert carrier or diluent, a hard gelatin capsule.

Any inert excipient that is commonly used as a carrier or diluent may be used in the formulations of the present invention, such as for example, a gum, a starch, a sugar, a cellulosic material, an acrylate, or mixtures thereof. A preferred diluent is microcrystalline cellulose. The compositions may further comprise a disintegrating agent (e.g., croscarmellose sodium) and a lubricant (e.g., magnesium stearate), and in addition may comprise one or more additives selected from a binder, a buffer, a protease inhibitor, a surfactant, a solubilizing agent, a plasticizer, an emulsifier, a stabilizing agent, a viscosity increasing agent, a sweetener, a film forming agent, or any combination thereof. Furthermore, the compositions of the present invention may be in the form of controlled release or immediate release formulations.

For liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil. Solutions or suspensions can also include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide.

In addition, the compositions may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCl, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol), a glidant (e.g., colloidal silicon dioxide), anti-oxidants (e.g., ascorbic acid, sodium metabisulfite, butylated hydroxyanisole), stabilizers (e.g., hydroxypropyl cellulose, hyroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates) and/or adjuvants.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The preparation of pharmaceutical compositions that contain an active component is well understood in the art, for example, by mixing, granulating, or tablet-forming processes. The active therapeutic ingredient is often mixed with excipients that are pharmaceutically acceptable and compatible with the active ingredient. For oral administration, the active agents are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions and the like as detailed above.

Those of ordinary skill in the art would be capable of determining dosing and dosing regimens for the different compounds appropriate for particular disease states and such dosing can be determined empirically. The amount of a compound of the invention necessary to achieve the desired biological effect depends on a number of factors, for example the specific compound chosen, the intended use, the mode of administration and the clinical condition of the patient.

The daily dose is generally in the range from 0.3 mg to 500 mg (typically from 3 mg to 50 mg) per day and per kilogram of bodyweight, for example 3-10 mg/kg/day. An intravenous dose may be, for example, in the range from 0.3 mg to 1.0 mg/kg, which can suitably be administered as slow infusion. Single doses may contain, for example, from 1 mg to 10 g of the active ingredient. Thus, ampoules for injections may contain, for example, from 1 mg to 100 mg, and single-dose formulations which can be administered orally, such as, for example, tablets or capsules, may contain, for example, from 0.05 to 1000 mg, typically from 0.5 to 500 mg.

The daily administration is then repeated continuously for a period of several days to several years. Oral treatment may continue for between one week and the life of the patient. Preferably the administration takes place for five consecutive days after which time the patient can be evaluated to determine if further administration is required. The administration can be continuous or intermittent, i.e., treatment for a number of consecutive days followed by a rest period. The compounds of the present invention may be administered intravenously on the first day of treatment, with oral administration on the second day and all consecutive days thereafter.

The amount of the compound administered to the patient is less than an amount that would cause toxicity in the patient. In the certain embodiments, the amount of the compound that is administered to the patient is less than the amount that causes a concentration of the compound in the patient's plasma to equal or exceed the toxic level of the compounds.

For the therapy of the abovementioned conditions, the compounds of formula (I) and (II) may be used as the compound itself, but they are preferably in the form of a pharmaceutical composition with an acceptable carrier. The carrier must, of course, be acceptable in the sense that it is compatible with the other ingredients of the composition and is not harmful for the patient's health. The carrier may be a solid or a liquid or both and is preferably formulated with the compound as a single dose, for example as a tablet, which may contain from 0.05% to 95% by weight of the active ingredient. Other pharmaceutically active substances may likewise be present, including other compounds of the invention. The pharmaceutical compositions of the invention can be produced by one of the known pharmaceutical methods, which essentially consist of mixing the ingredients with pharmacologically acceptable carriers and/or excipients.

Pharmaceutical compositions of the invention are those suitable for oral, rectal, topical, peroral (for example sublingual), intraperitoneal and parenteral (for example subcutaneous, intramuscular, intradermal or intravenous) administration, although the most suitable mode of administration depends in each individual case on the nature and severity of the condition to be treated and on the nature of the compound of formula (I) or (II) used in each case. Coated formulations and coated slow-release, PEG, liposomal formulations also belong within the framework of the invention.

Suitable pharmaceutical compounds for oral administration may be in the form of separate units such as, for example, capsules, cachets, chewable tablets or tablets, each of which contain a defined amount of the compound of formula (I) or (II); as powders or granules; as solution or suspension in an aqueous or non aqueous liquid; or as an oil-in-water or water-in-oil emulsion. These compositions may, as already mentioned, be prepared by any suitable pharmaceutical method which includes a step in which the active ingredient and the carrier (which may consist of one or more additional ingredients) are brought into contact. The compositions are generally produced by uniform and homogeneous mixing of the active ingredient with a liquid and/or finely divided solid carrier, after which the product is shaped if necessary.

Pharmaceutical compositions which are suitable for peroral (sublingual) administration comprise chewable tablets which contain a compound of formula (I) or (II) with a flavoring, normally sucrose and gum arabic, and pastilles which comprise the compound in an inert base such as gelatin and glycerol or sucrose and gum arabic.

Pharmaceutical compositions suitable for parenteral administration comprise preferably sterile aqueous preparations of one or more of the compounds of general formulae (I) and (II), which are preferably isotonic with the blood of the intended recipient. These preparations are preferably administered intravenously, although administration may also take place by subcutaneous, intramuscular or intradermal injection. These preparations can preferably be produced by mixing the compound with water and making the resulting solution sterile and isotonic with blood. Injectable compositions of the invention generally contain from 0.1 to 5% by weight of the active compound.

Pharmaceutical compositions suitable for rectal administration are preferably in the form of single-dose suppositories. These can be produced by mixing one or more of the compounds of general formulae (I) and (II) with one or more conventional solid carriers, for example cocoa butter, and shaping the resulting mixture.

Pharmaceutical compositions suitable for topical use on the skin are preferably in the form of ointment, cream, lotion, paste, spray, aerosol or oil. Carriers which can be used are petrolatum, lanolin, polyethylene glycols, alcohols and combinations of two or more of these substances. The active ingredient is generally present in a concentration of from 0.1 to 15% by weight of the composition, for example from 0.5 to 2%.

Transdermal administration is also possible. Pharmaceutical compositions suitable for transdermal uses. A suitable active ingredient concentration is about 1% to 35%, preferably about 3% to 15%.

The compounds of the formulae I and II are distinguished by favorable effects on metabolic disorders. They beneficially influence lipid and glucose metabolism, in particular they lower the triglyceride level and are suitable for the prevention and treatment of type II diabetes and arteriosclerosis and the diverse complications thereof.

The compounds and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

### EXAMPLE 1

### Assay for Potency of the Inhibitors of HSL Activity

Production of Recombinant HSL: Recombinant His-HSL was generated by cloning full-length rat HSL cDNA into the SmaI site of pAcHLT-A containing a His6 tag. pAcHLT-A-HSL (5 μg) was co-transfected into Sf21 cells with 0.5 μg of BaculoGold™ DNA using the transfection kit from the manufacturer. The titer of the recombinant virus was determined using an end point dilution assay, and the virus was re-amplified to a final titer of 1.5×10⁷ pfu/ml. To produce recombinant proteins, Sf21 cells were grown in 150 mm Petri dishes and each 2×10⁷ cells were infected with 100 μl of the high titer recombinant virus; cells were harvested three days after infection. After harvesting and cell extraction, His-HSL was purified on a Ni-agarose column.

Preparation of Substrate for Neutral Cholesteryl Ester Hydrolase Activity Assay: HSL activity was determined as neutral cholesteryl ester hydrolase activity using a cholesteryl[1-14C]oleate emulsion for measurement of cellular activity, as described previously. Substrate for the cholesteryl ester assay was prepared by adding 1.25 μCi cholesteryl [1-14C]oleate (purified by thin-layer chromatography), 0.043 mmol phosphatidylcholine, and 0.011 mmol cholesteryl oleate into 2.5 mL of 100 mM potassium phosphate buffer (pH 7.0) containing 5 mM sodium taurocholate. The substrate solution was vortexed and then sonicated for 3 hours with a Branson Sonifier/Cell Disruptor model W-350 on an output setting of 5.0 (50%). The substrate was centrifuged at 3000 rpm for 15 min to remove metallic fragments released by the probe and stored under nitrogen at 4° C. for up to one week.

Preparation of Inhibitor Stock Solution: Inhibitors (fraction # O-4355, O-4350, O-4361, O-4456, O-4457) were obtained from olives, olive oil, roots, bark and leaves from olea europeae species as previously described (Paiva-Martins et al., J. Agric. Food Chem., 49:4214-4219, 2001) and dissolved in 200 μL DMSO with 800 μL distilled water at a concentration of 10 mg/ml. This was regarded as stock solution. 0-60 μL of the stock solution were added to the HSL assay to achieve a final concentration of 0-2.4 mg/ml of the inhibitors. The same volume of DMSO without inhibitors was performed as a control.

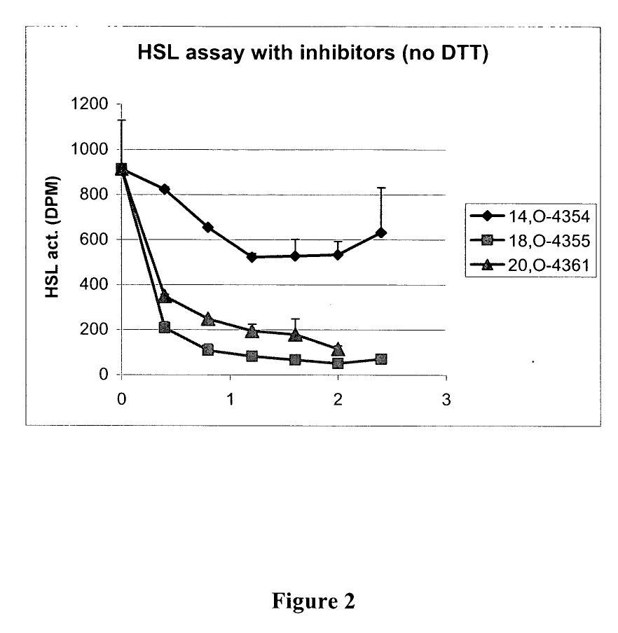

Assay for Potency of Inhibition on HSL Activity by the Inhibitors: Aliquots of recombinant HSL protein and various concentrations of inhibitors (0-2.4 mg/mL final concentration) were mixed and the volume adjusted to 100 μL and mixed with 140 μL 0.05% bovine serum albumin in 100 mM potassium phosphate, pH 7.0. After the addition of 10 μL of substrate (100 μM final concentration), the assay was carried out at 37° C. for 60 min. The reaction was stopped by the addition of chloroform:methanol:heptane (250:230:180). After the addition of borate/carbonate buffer (0.1 M, pH, 10.5), the tubes were vortexed and centrifuged, and aliquots of the upper phase were taken for liquid scintillation counting in a Beckman scintillation counter. The results set forth in FIG. 2 indicate that fraction O-4355 demonstrated the most anti-HSL activity while fractions O-4354 and O-4361 demonstrated some anti-HSL activity.

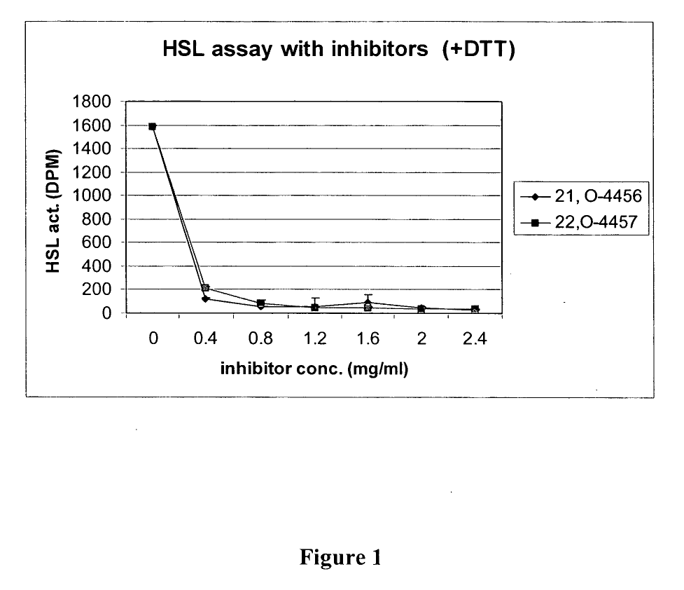

HPLC/MS and NMR studies were then conducted (data not shown) to identify the active single molecules of fraction O-4355. These single molecules arep-HPEA-EDA (fraction O-4456) and 3,4-DHPEA-EDA (fraction O-4457). The results, set forth in FIG. 1 and Table 1, indicate that fractions O-4456 and O-4457 inhibit HSL by 98% and 97%, respectively.

TABLE 1

| | O-4456 | HSL Activity | | O-4457 | HSL Activity | |
|---|---|---|---|---|---|---|
| | Concentration | (DPM) | Standard | Concentration | (DPM) | Standard |
| | (mg/mL) | (average) | Deviation | (mg/mL) | (average) | Deviation |
| HHSL + | 0 | 1585.64 | 14.84924 | 0 | 1585.64 | 14.84924 |
| DTT (1 mM) | 0.4 | 123.52735 | 10.72349 | 0.4 | 214.85 | 12.09153 |
| | 0.8 | 52.6035 | 0.021213 | 0.8 | 87.105 | 24.64267 |
| | 1.2 | 51.145 | 80.6031 | 1.2 | 48.025 | 6.102332 |
| | 1.6 | 88.595 | 73.53911 | 1.6 | 49.885 | 5.591347 |
| | 2.0 | 49.56 | 2.333452 | 2.0 | 39.545 | 1.576848 |
| | 2.4 | 25.035 | 4.688118 | 2.4 | 32.495 | 7.007428 |

| | O-4456 | O-4457 |
|---|---|---|
| % inhibition | 98.421142 | 97.95067 |

The experiment above was also performed using oleuropein and hydroxytyrosol as inhibitors. Results indicated that these compounds did not demonstrate anti-HSL activity.

### EXAMPLE 2

### Assay for Potency of Other Inhibitors of HSL Activity

The experiment provided in Example 1 is repeated with other potential inhibitors (e.g., compounds of the invention) to assess the inhibitor's affect on HSL activity.

Production of Recombinant HSL: Recombinant His-HSL is generated by cloning full-length rat HSL cDNA into the SmaI site of pAcHLT-A containing a His6 tag. pAcHLT-A-HSL is co-transfected into Sf21 cells with BaculoGold™ DNA using the transfection kit from the manufacturer. The titer of the recombinant virus is determined using an end point dilution assay. To produce recombinant proteins, Sf21 cells are grown in Petri dishes and cells are infected with the high titer recombinant virus and harvested three days after infection. After harvesting and cell extraction, His-HSL is purified on a Ni-agarose column.

Preparation of Substrate for Neutral Cholesteryl Ester Hydrolase Activity Assay: HSL activity is determined as neutral cholesteryl ester hydrolase activity using a cholesteryl[14C]oleate emulsion for measurement of cellular activity, as described previously. A substrate for the cholesteryl ester assay is prepared, vortexed and then sonicated for 3 hours with a Branson Sonifier/Cell Disruptor model W-350 on an output setting of 5.0 (50%). The substrate is then centrifuged to remove metallic fragments released by the probe and stored under nitrogen at 4° C.

Preparation of Inhibitor Stock Solution: Inhibitors (e.g., fraction # O-4477) are obtained from olives, olive oil, roots, bark and leaves from olea europeae species as previously described (Paiva-Martins et al., J. Agric. Food Chem., 49:4214-4219, 2001) and dissolved in DMSO with distilled water. This is regarded as stock solution. 0-60 μL of the stock solution are added to the HSL assay to achieve a final concentration of 0-2.4 mg/ml of the inhibitors. The same volume of DMSO without inhibitors is performed as a control.

Assay for Potency of Inhibition on HSL Activity by the Inhibitors: Aliquots of recombinant HSL protein and various concentrations of inhibitors are mixed and the volume adjusted to 100 μL. After the addition of the substrate, the assay is carried out at 37° C. for 60 min. The reaction is stopped by the addition of chloroform:methanol:heptane (250:230:180). After the addition of borate/carbonate buffer (0.1 M, pH 10.5), the tubes are vortexed and centrifuged, and aliquots of the upper phase are taken for liquid scintillation counting in a Beckman scintillation counter.

It is expected that the compound(s) of formula (I) will demonstrate anti-HSL activity.

### EXAMPLE 3

### Assay for Potency of Other Inhibitors of HSL Activity

The experiment provided in Example 1 was repeated with other potential inhibitors (e.g., fraction # O-4356, O-4357, O-4358, O-4359, O-4360, O-4349, O-4351, O-4352, O-4353, O-4363, O-4362, O-4364, O-4365, O-4354, O-4348, O-4366, O-4367, O-4355, O-4350 and O-4361) to assess the inhibitor's affect on HSL activity.

Production of Recombinant HSL: Recombinant His-HSL was generated by cloning full-length rat HSL cDNA into the SmaI site of pAcHLT-A containing a His6 tag. pAcHLT-A-HSL is co-transfected into Sf21 cells with BaculoGold™ DNA using the transfection kit from the manufacturer. The titer of the recombinant virus was determined using an end point dilution assay. To produce recombinant proteins, Sf21 cells were grown in Petri dishes and cells were infected with the high titer recombinant virus and harvested three days after infection. After harvesting and cell extraction, His-HSL was purified on a Ni-agarose column.

Preparation of Substrate for Neutral Cholesteryl Ester Hydrolase Activity Assay: HSL activity was determined as neutral cholesteryl ester hydrolase activity using a cholesteryl[14C]oleate emulsion for measurement of cellular activity, as described previously. A substrate for the cholesteryl ester assay was prepared, vortexed and then sonicated for 3 hours with a Branson Sonifier/Cell Disruptor model W-350 on an output setting of 5.0 (50%). The substrate was then centrifuged to remove metallic fragments released by the probe and stored under nitrogen at 4° C.

Preparation of Inhibitor Stock Solution: Inhibitors (e.g., fraction # O-4356, O-4357, O-4358, O-4359, O-4360, O-4349, O-4351, O-4352, O-4353, O-4363, O-4362, O-4364, O-4365, O-4354, O-4348, O-4366, O-4367, O-4355, O-4350, O-4361 and O-4477) were obtained from olives, olive oil, roots, bark and leaves from olea europeae species as previously described (Paiva-Martins et al., J. Agric. Food Chem., 49:4214-4219, 2001) and dissolved in DMSO with distilled water. This was regarded as stock solution. 0-60 μL of the stock solution was added to the HSL assay to achieve a final concentration of 0-2.4 mg/ml of the inhibitors. The same volume of DMSO without inhibitors was performed as a control.

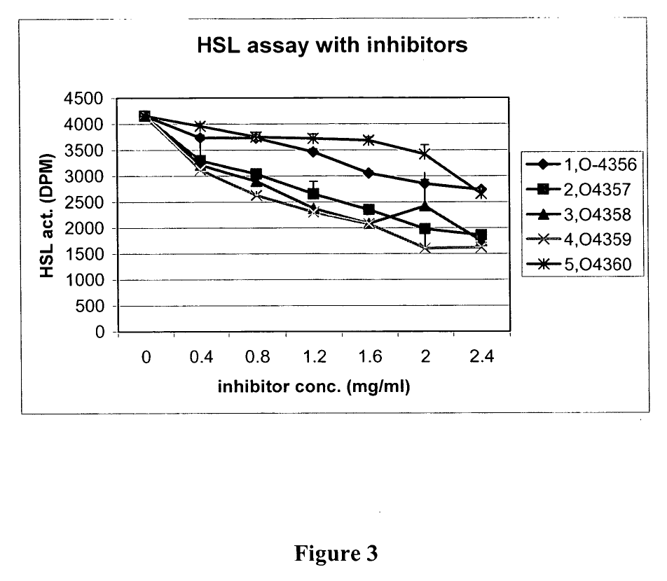

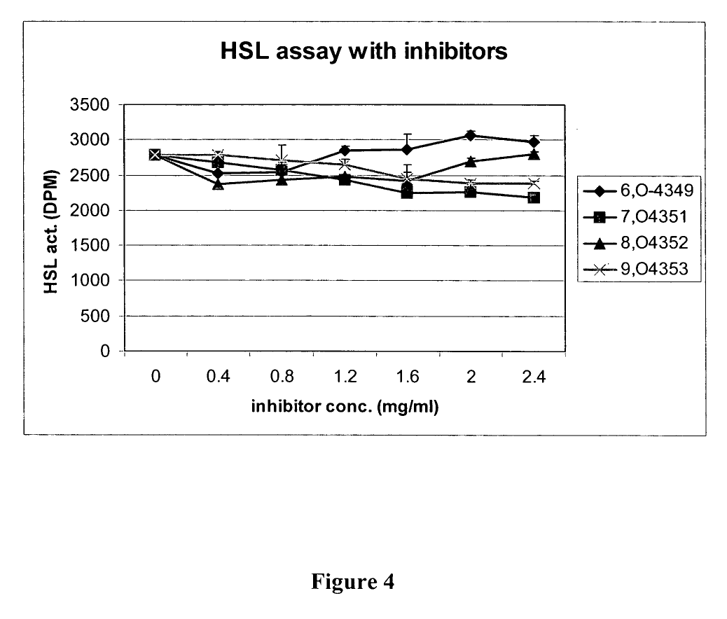

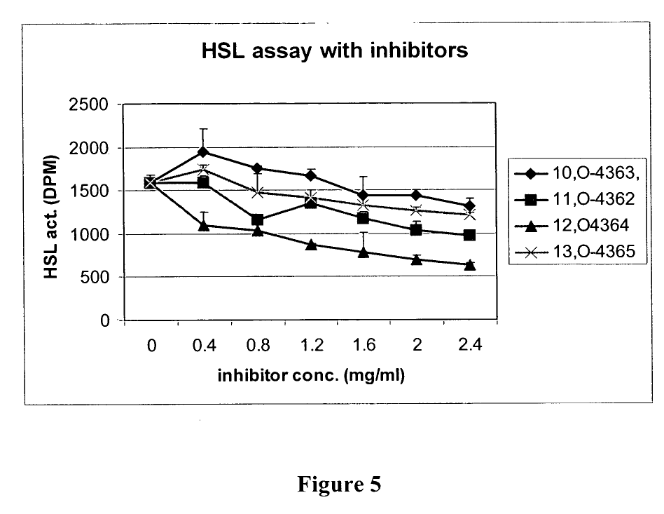

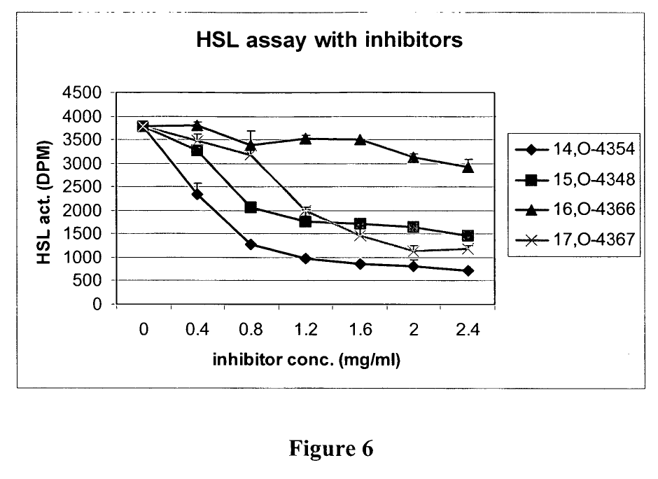

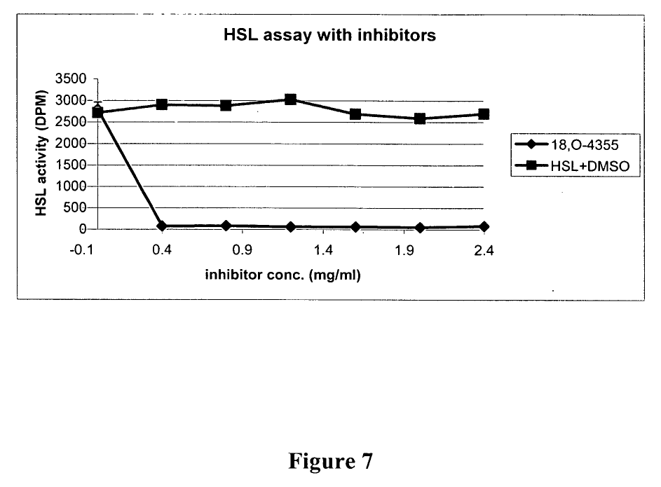

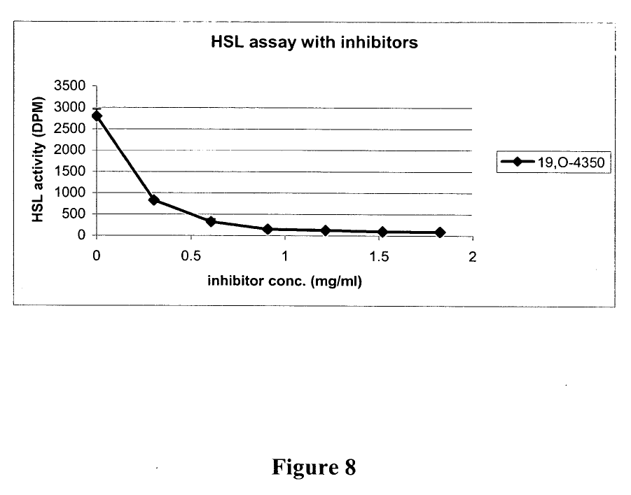

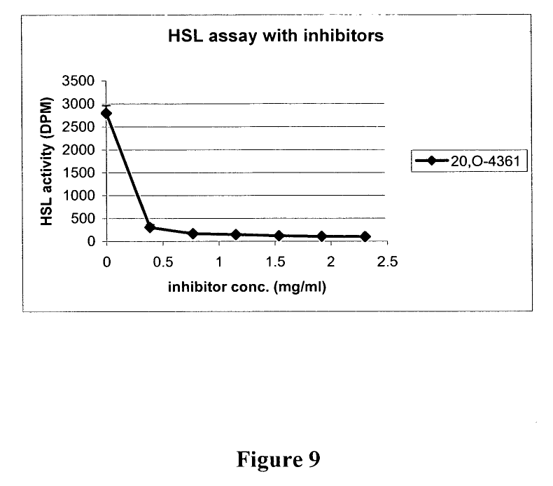

Assay for Potency of Inhibition on HSL Activity by the Inhibitors: Aliquots of recombinant HSL protein and various concentrations of inhibitors were mixed and the volume adjusted to 100 μL. After the addition of the substrate, the assay was carried out at 37° C. for 60 min. The reaction was stopped by the addition of chloroform:methanol:heptane (250:230:180). After the addition of borate/carbonate buffer (0.1 M, pH 10.5), the tubes were vortexed and centrifuged, and aliquots of the upper phase were taken for liquid scintillation counting in a Beckman scintillation counter. The results are set forth in FIGS. 3-9 and Table 2.

TABLE 2

| | Fraction | | | | |
|---|---|---|---|---|---|
| | O-4356 | O-4357 | O-4358 | O-4359 | O-4360 |
| % inhibition | 34.25102 | 55.38591 | 58.06684 | 61.01226 | 36.13849 |
| | Fraction | | | | |
| | O-4349 | O-4351 | O-4352 | O-4353 | O-4363 |
| % inhibition | −6.77662 | 21.49516 | −0.73503 | 14.73646 | 16.90319 |

| | Fraction | | | | |
|---|---|---|---|---|---|
| | O-4362 | O4364 | O-4365 | O-4354 | O-4348 |
| % inhibition | 38.66288 | 60.54872 | 23.65184 | 81.20032 | 61.354 |

| | Fraction | | | | |
|---|---|---|---|---|---|
| | O-4366 | O-4367 | O-4355 | , O-4350 | O-4361 |
| % inhibition | 22.64176 | 68.60095 | 97.12294 | 96.71194 | 96.47963 |

### EXAMPLE 4

### Preparation of Further HSL Activity Inhibitors

This example illustrates procedures for the preparation of intermediates and methods for the preparation of products according to this invention.

Analytical HPLC was performed using an Apollo C₁₈ 150 mm×4.6 mm/5 μm column coupled with an Agilent 1050 series VWD UV detector at 254 nm. Conditions: Solvent A: H₂O/0.1% TFA; Solvent B: acetonitrile, flow rate 1.5 mL/min.

Proton magnetic resonance (¹H NMR) spectra were recorded on either a Varian INOVA 400 MHz (¹H) NMR spectrometer or a Varian INOVA 500 MHz (¹H) NMR spectrometer. All spectra were determined in the solvents indicated. Although chemical shifts are reported in ppm downfield of tetramethylsilane, they are referenced to the residual proton peak of the respective solvent peak for ¹H NMR. Interproton coupling constants are reported in Hertz (Hz).

LCMS spectra were obtained using a ThermoFinnigan AQA MS ESI instrument utilizing a Phenomenex Aqua 5 micron C₁₈ 125 Å 50×4.60 mm column. The spray setting for the MS probe was at 350 μL/min with a cone voltage at 25 mV and a probe temperature at 450° C. The spectra were recorded using ELS and UV (254 nm) detection.

Silica gel chromatography was carried out on a Teledyne ISCO CombiFlash Companion Flash Chromatography System with a variable flow rate from 5-100 mL/min. The columns used were Teledyne ISCORediSep Disposable Flash Columns (4, 12, 40, 80, or 120 g prepacked silica gel), which were run with a maximum capacity of 1 g crude sample per 10 g silica gel. Samples were preloaded on Celite in Analogix Sample Loading Cartridges with frits (1/in, 1/out). Peaks were detected by variable wavelength UV absorption (200-360 nm). The resulting fractions were analyzed, combined as appropriate, and evaporated under reduced pressure to provide purified material.

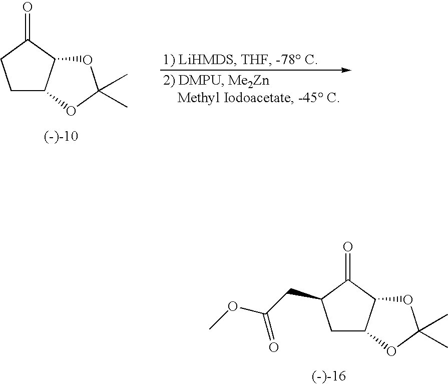

((3aR,5S,6aR)-2,2-Dimethyl-4-oxotetrahydrocyclopenta[1,3]dioxol-5-yl)acetic acid methyl ester.

A solution of LiHMDS (1 M in THF, 70.43 mL, 1.1 eq) in THF (350 mL) was cooled to −78° C. and a solution of enone (−)-10 (10 g, 0.0640 mol) in THF (250 mL) was slowly added by cannula. After one hour, DMPU (23.2 mL, 3 eq) was added, followed by dimethylzinc (2 M in toluene, 32 mL, 1 eq). After 30 minutes, freshly prepared methyl iodoacetate (38.41 g, 3 eq) in THF (150 mL) and cooled to −78° C. was added by cannula and the reaction was allowed to warm to −45° C. After four hours at −45° C., the reaction was quenched with saturated aqueous ammonium chloride (˜500 mL). The mixture was diluted with additional water and the layers were separated. The aqueous layer was extracted with ether (3×300 mL). The organic fractions were combined, dried over sodium sulfate, filtered and concentrated. The material was purified by silica gel flash chromatography (O-30% ethyl acetate in heptane, linear gradient) to provide 9.32 g (64% yield) of ester (−)-16 as a yellowish oil. ¹H NMR (400 MHz, CHLOROFORM-d) δ ppm 4.83 (t, J=4.83 Hz, 1H) 4.33 (d, J=5.32 Hz, 1H) 3.67 (s, 3H) 2.80-2.97 (m, 1H) 2.69 (d, J=5.27 Hz, 2H) 2.36-2.52 (m, 1H) 1.80-1.95 (m, 1H) 1.43 (s, 3H) 1.36 (s, 3H).

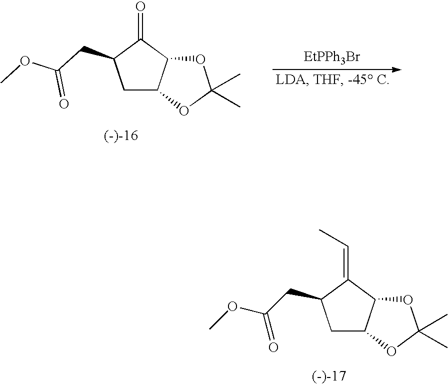

t [(3aS,5S,6aR)-4-Eth-(E)-ylidene-2,2-dimethyltetrahydrocyclopenta-[1,3]-dioxol-5-yl]-acetic acid methyl ester.

To a suspension of ethyl triphenylphosphonium bromide (7.2 g, 19.3 mmol) in THF (50 mL) at 0° C. was added LDA (2 M in THF, 8.7 mL). The resulting red solution was stirred at 0° C. for 30 minutes and then for an hour at room temperature. The reaction was then cooled to −45° C. and a −78° C. solution of (−)-16 (1 g, 4.4 mmol) in THF (30 mL) was added slowly by cannula. The reaction was stirred at −45° C. for two hours. After this time the reaction was quenched with ammonium chloride (1 mL) and adsorbed on to Celite. The reaction was purified by silica gel flash chromatography (0-30% ethyl acetate in heptane, linear gradient) to provide 302 mg (29% yield) of alkene (−)-17 in a greater than 10:1 E:Z ratio as a clear oil. ¹H NMR (400 MHz, CHLOROFORM-d) δ ppm 5.64-5.82 (m, 1H) 4.79 (dd, J=5.74, 1.15 Hz, 1H) 4.55-4.71 (m, 1H) 3.67 (s, 3H) 3.34 (br. s., 1H) 2.60 (dd, J=15.37, 4.69 Hz, 1H) 2.29 (dd, J=15.42, 9.66 Hz, 1H) 2.04-2.19 (m, 1H) 1.73-1.88 (m, 1H) 1.67-1.73 (m, 3H) 1.44 (s, 3H) 1.33 (s, 3H).

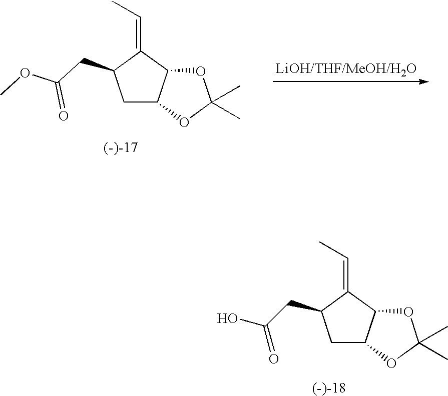

To a stirring solution of methyl ester (−)-17 (33.4 mg, 0.14 mmol) in a 1:1 mixture of THF/MeOH (0.8 mL each) was added a freshly prepared aqueous lithium hydroxide solution (1 M, 0.556 mL). After 1.5 hours the reaction was diluted with saturated aqueous ammonium chloride. The pH of the solution was found to be ˜6, and the solution was extracted with ethyl acetate (3×50 mL). The organic fractions were combined, dried over sodium sulfate, filtered, and concentrated to provide an amber oil (28 mg, 89% yield). The stereochemistry of the alkene was confirmed by NOE spectroscopy. ¹H NMR (400 MHz, CHLOROFORM-d) δ ppm 5.65-5.82 (m, 1H) 4.79 (dd, J=5.59, 1.10 Hz, 1H) 4.57-4.69 (m, 1H) 3.35 (br. s., 1H) 2.66 (dd, J=15.74, 4.51 Hz, 1H) 2.32 (dd, J=15.72, 9.76 Hz, 1H) 2.08-2.23 (m, 1H) 1.76-1.88 (m, 1H) 1.66-1.76 (m, 3H) 1.44 (s, 3H) 1.33 (s, 3H).

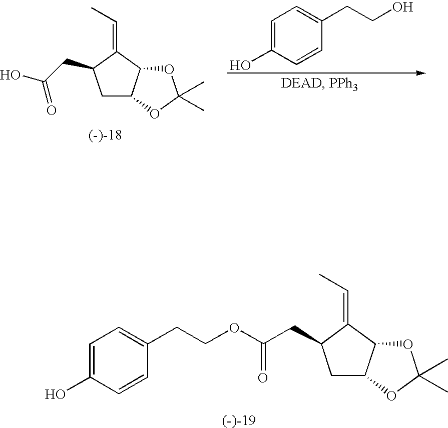

[(3aS,5S,6aR)-4-Eth-(E)-ylidene-2,2-dimethyltetrahydrocyclopenta[1,3]dioxol-5-yl]-acetic acid 2-(4-hydroxy-phenyl)-ethyl ester

To a solution of (−)-18 (325 mg, 1.4 mmol) in dry THF (50 mL) at 0° C. were sequentially added triphenylphosphine (1.51 g, 5.8 mmol), 4-hydroxyphenethyl alcohol (200 mg, 1.4 mmol), and diethylazodicarboxylate (0.9 mL, 5.8 mmol). The reaction was stirred for 16 hours, gradually warming to room temperature. The reaction was concentrated, and the product was purified by silica gel chromatography (0-30% ethyl acetate in heptane, linear gradient). The title compound was isolated as a clear oil (425 mg, 85%). ¹H NMR (400 MHz, CHLOROFORM-d) δ ppm 1.34 (s, 3H) 1.45 (s, 3H) 1.68 (dt, J=7.18, 1.29 Hz, 3 H) 1.74 (dt, J=14.06, 5.86 Hz, 1H) 2.01-2.11 (m, 1H) 2.26 (dd, J=15.42, 9.76 Hz, 1H) 2.58 (dd, J=15.33, 4.59 Hz, 1H) 2.86 (t, J=7.03 Hz, 2H) 3.32 (br. s., 1H) 4.25 (t, J=7.03 Hz, 2H) 4.60 (td, J=5.86, 3.71 Hz, 1H) 4.77 (dd, J=5.66, 1.17 Hz, 1H) 5.30 (br. s., 1H) 5.66-5.76 (m, 1H) 6.77 (ddd, J=8.98, 2.93, 2.54 Hz, 2H) 7.07 (ddd, J=9.08, 2.73, 2.44 Hz, 2H)., 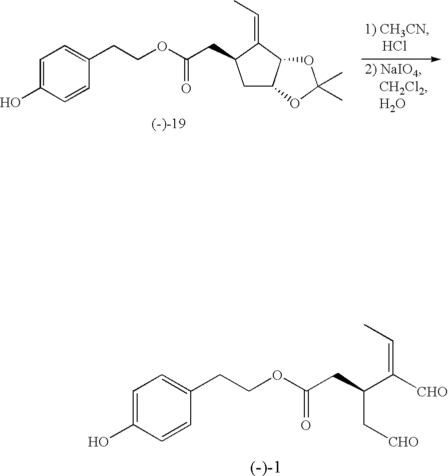
(−)-Oleocanthal.

To a solution of (−)-19 (200 mg, 0.6 mmol) in dry acetonitrile (6 mL) was added aqueous hydrochloric acid (4 M, 2 mL). The solution was stirred at room temperature for 1 hour. The reaction was diluted with ethyl acetate and the aqueous layer was separated. The aqueous portion was extracted with ethyl acetate (3×10 mL). The combined organic portions were washed with brine (5 mL), dried over sodium sulfate, and concentrated to dryness. The product was purified by silica gel flash chromatography (0-10% MeOH in dichloromethane, linear gradient), yielding the diol as a clear oil (112 mg, 63%).

The diol product was immediately dissolved in dichloromethane (6 mL) and cooled to 0° C. A solution of sodium periodate (110 mg, 0.5 mmol) was added, and the reaction was allowed to stir at 0° C. for 75 minutes. The reaction was diluted with dichloromethane (10 mL). The aqueous layer was separated and extracted with additional dichloromethane (3×10 mL). The combined organic portions were washed with brine (5 mL), dried over sodium sulfate, and concentrated in vacuo. Purification was achieved by filtration through a short plug of silica gel, eluting with 30% ethyl acetate in heptane. The title compound was isolated as a clear oil (50 mg, 45%). ¹H NMR (400 MHz, CHLOROFORM-d) δ ppm 2.08 (d, J=7.03 Hz, 3H) 2.52-2.79 (m, 1H) 2.66 (dd, J=11.52, 7.61 Hz, 2H) 2.83 (t, J=6.93 Hz, 2H) 2.99 (ddd, J=18.35, 8.59, 1.17 Hz, 1H) 3.56-3.68 (m, 1H) 4.14-4.28 (m, 2H) 6.64 (q, J=7.16 Hz, 1H) 6.77 (d, J=8.59 Hz, 2H) 7.05 (d, J=8.40 Hz, 2H) 9.24 (d, J=2.15 Hz, 1H) 9.63 (t, J=0.98 Hz, 1H).,

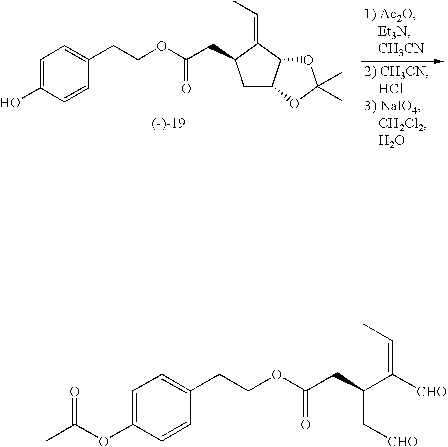

### (E)-(S)-4-Formyl-3-(2-oxoethyl)-hex-4-enoic acid 2-(4-acetoxyphenyl)-ethyl ester

To a solution of (−)-19 (50 mg, 0.1 mmol) in dry acetonitrile (2 mL) was added an excess of triethylamine (0.03 mL, 0.4 mmol) and acetic anhydride (0.03 mL, 0.3 mmol). The solution was stirred at room temperature for 30 minutes. The reaction was then diluted with ethyl acetate (10 mL) and washed with water (2 mL) and brine (2 mL). The organic portion was concentrated to dryness in vacuo.

The resultant white solid was dissolved in acetonitrile (2 mL) and aqueous hydrochloric acid (1 M, 1 mL) was added. The solution was stirred at room temperature for 1 hour. The reaction was then diluted with ethyl acetate (10 mL) and washed with water (2 mL) and brine (2 mL). The organic portion was concentrated to dryness in vacuo.

The resulting product was dissolved in dichloromethane (2 mL) and cooled to 0° C. A solution of sodium periodate (45 mg, 0.2 mmol) in water (1 mL) was added, and the mixture was vigorously stirred at 0° C. for 1 hour. The reaction was then diluted with dichloromethane (10 mL) and washed with water (1 mL) and brine (2 mL). The organic portion was concentrated to dryness in vacuo. Filtration through a short plug of silica gel with 30% ethyl acetate in heptane as eluent afforded the title compound as a colorless oil (19 mg, 38% yield for three steps). ¹H NMR (400 MHz, CHLOROFORM-d) δ ppm 2.07 (d, J=7.03 Hz, 3H) 2.30 (s, 3H) 2.56-2.80 (m, 3H) 2.90 (t, J=6.83 Hz, 2H) 2.98 (ddd, J=18.35, 8.59, 1.17 Hz, 1H) 3.56-3.68 (m, 1H) 4.18-4.29 (m, 2H) 6.63 (q, J=7.03 Hz, 1 H) 7.02 (d, J=8.59 Hz, 2H) 7.20 (d, J=8.59 Hz, 2H) 9.23 (d, J=1.95 Hz, 1H) 9.64 (t, J=0.98 Hz, 1H).

All other 4-substituted phenolic esters were prepared in similar fashion, using the appropriate acid anhydride or acid chloride:

TABLE 3
Drug Characterization

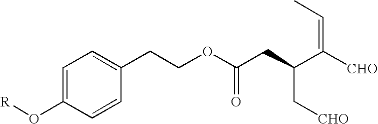

| R= | ¹H-NMR Diagnostic Peaks | HPLC Retention Time^{a} | TLC Retention Factor^{b} | MS Result |
|---|---|---|---|---|
| 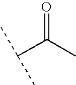 | 2.30 (s, 3 H) 7.02 (d, J=8.59 Hz, 2 H) 7.20 (d, J=8.59 Hz, 2 H) 9.23 (d, J=1.95 Hz, 1 H) 9.64 (t, J=0.98 Hz, 1 H) | 11.7 minutes | 0.36 | 328 (M − OH)⁺ |
| 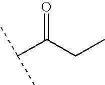 | 1.27 (t, J=7.52 Hz, 3 H) 2.59 (q, J=7.61 Hz, 2 H) 7.01 (d, J=8.59 Hz, 2 H) 7.20 (d, J=8.59 Hz, 2 H) 9.22 (d, J=1.95 Hz, 1 H) 9.64 (s, 1 H) | 12.9 minutes | 0.43 | 343 (M − OH)+361 (M + H)+ |
| 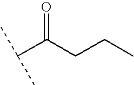 | 7.13 (d, J=8.59 Hz, 2 H), 7.24 (d, J=8.4 Hz, 2 H), 9.24 (d, J=1.95 Hz, 1 H), 9.64 (t, J=0.98 Hz, 1 H) | 14.2 minutes | 0.36 | 357 (M − OH)+ |
| 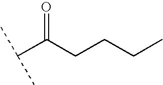 | 2.46 (t, J=7.42 Hz, 2 H), 7.01 (d, J=8.40 Hz, 2 H) 7.19 (d, J=8.40 Hz, 2 H) 9.22 (d, J=1.95 Hz, 1 H) 9.64 (t, J=0.98 Hz, 1 H) | 15.4 minutes | 0.46 | 371 (M − OH)+389 (M + H)+ |
| 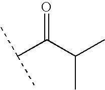 | 7.00 (d, J=8.79 Hz, 2 H) 7.19 (d, J=8.59 Hz, 2 H) 9.22 (d, J=1.95 Hz, 1 H) 9.64 (t, J=1.07 Hz, 1 H) | 14.1 minutes | 0.46 | 357 (M − OH)+375 (M + H)+ |
| 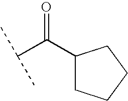 | 6.99 (d, J=8.59 Hz, 2 H), 7.18, (d, J=8.59 Hz, 2 H), 9.21 (d, J=1.95 Hz, 1 H), 9.63 (t, J=1.07 Hz, 1 H) | 15.6 minutes | 0.38 | 383 (M − OH)+ |
| 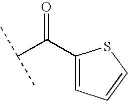 | 7.25 (d, J=8.59 Hz, 2 H), 7.83 (dd, J=4.98, 1.27 Hz, 1 H) 9.24 (d, J=1.95 Hz, 1 H) 9.65 (s, 1 H) | 13.6 minutes | 0.41 | 397 (M − OH)⁺ |
| 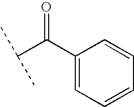 | 7.12-7.20 (m, 2H), 7.23-7.28 (m, 2H), 7.49-7.56 (m, 2H), 7.62-7.68 (m, 1H), 8.21 (dd, J=8.40, 1.37 Hz, 2H), 9.24 (d, J=1.95 Hz, 1H), 9.66 (s, 1H). | 15.3 minutes | 0.47 | 391 (M − OH)⁺ |
| 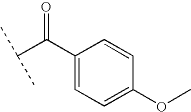 | 7.00 (d, 2 H), 7.18 (d, 8.79 Hz, 2 H), 9.21 (d, J=1.95 Hz, 1 H), 9.63 (t, J=0.98 Hz, 1 H) | 14.2 minutes | 0.35 | 421 (M − OH)⁺ |
| 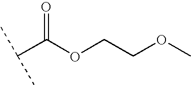 | 3.44 (s, 3H), 3.66-3.74 (m, 2H), 4.37-4.44 (m, 2H), 7.09-7.15 (m, 2H), 7.18-7.24 (m, 2H), 9.23 (d, J=1.95 Hz, 1H), 9.65 (s, 1H) | 11.9 minutes | 0.26 | 389 (M − OH)^{+407 (M + H)+} |

^{a}HPLC gradient was 10% to 90% Acetonitrile in water (with 0.1% TFA modifier) over 20 minutes.
^{b}TLC samples were run on glass plates precoated with silica gel (250 μM) using 1:1 ethyl acetate/heptane as eluent.

Numerous modifications and variations in the practice of the invention are expected to occur to those skilled in the art upon consideration of the presently preferred embodiments thereof. Consequently, the only limitations which should be placed upon the scope of the invention are those which appear in the appended claims.

What is claimed is:

1. An isolated and purified compound comprising the formula set forth in formulae (I) and (II), and salts, solvates and hydrates, racemates, racemic mixtures and pure enantiomers, diastereomers, homologs, analogs and mixtures thereof, 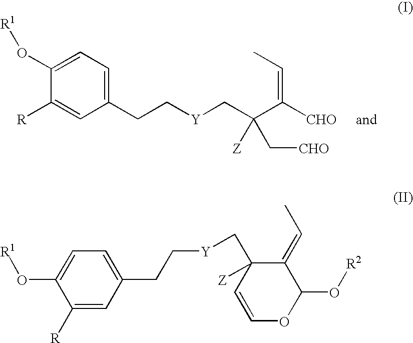
wherein R is hydrogen, hydroxyl or —OR¹;
wherein R¹ is hydrogen or a chemical moiety that can be cleaved in vivo to release a hydroxyl group;
wherein R² is hydrogen or a hydroxyl protecting group, —SO₃H and —SO₃R³ where R³ is a saturated or unsaturated aliphatic group; a substituted or unsubstituted saturated or unsaturated alicyclic group;
wherein R³ is a saturated or unsaturated aliphatic group, substituted or unsubstituted aliphatic group, substituted or unsubstituted saturated or unsaturated alicyclic group, substituted or unsubstituted aromatic group, substituted or unsubstituted heteroaromatic group, substituted or unsubstituted heterocyclic group, glucuronide or glucuronide ester, —P(O)(OH)₂, and —P(O)(OR³)₂;
wherein Y is —(CH₂)ₙ— where m=1 or 2, —OC(O)—, —O(CH₂)ₘ— where m=1 or 2, or —S(O)ₙ(CH₂)ₘ— where m=1 or 2; n=0, 1 or 2; and,
wherein Z is hydrogen, CH₃, F, Cl, Br or I.
wherein when Y is —OC(O)—, when Z is H and when R is hydrogen, R¹ is not hydrogen; and,
wherein when Y is —OC(O)—, when Z is H and when R is hydroxyl, R¹ is not hydrogen.

2. An isolated and purified compound according to claim 1 wherein R¹ is aliphatic or aromatic acyl, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, or a substituted or unsubstituted heterocyclic group, a carbalkoxy, a carbaryloxy, —SO₃H and —SO₃R³, —P(O)(OH)₂, and —P(O)(OR³)₂.

3. A pharmaceutical composition comprising one or more of the isolated and purified compounds of formulae (I) and (II) and a pharmaceutically acceptable carrier.

4. A method of inhibiting hormone sensitive lipase in a patient in need thereof comprising administering one or more of the isolated and purified compounds of formulae (I) and (II) 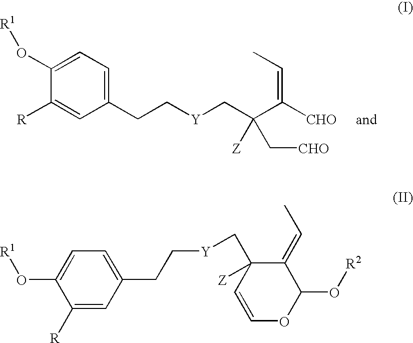
wherein R is hydrogen, hydroxyl or —OR¹;
wherein R¹ is hydrogen or a chemical moiety that can be cleaved in vivo to release a hydroxyl group′
wherein R² is hydrogen or a hydroxylprotecting group, —SO₃H and —SO₃R³ where R³ is a saturated or unsaturated aliphatic group; a substituted or unsubstituted saturated or unsaturated alicyclic group;
wherein R³ is a saturated or unsaturated aliphatic group, substituted or unsubtituted aliphatic group, substituted or unsubstituted saturated or unsaturated alicyclic group, substituted or unsubstituted aromatic group, substituted or unsubstituted heteroaromatic group, substituted or unsubstituted heterocyclic group, glucuronide or glucuronide ester, —P(O)(OH)₂, and —P(O)(OR³) 2;
wherein Y is —(CH₂)ₘ— where m=1 or 2, —OC(O)—, —O(CH₂)ₘ— where m=1 or 2, or —S(O)ₙ(CH₂)ₘ— where m=1 or 2; n=0, 1 or 2; and,
wherein Z is hydrogen, CH₃, F, Cl, Br or I.

5. The method according to claim 4 wherein R¹ is aliphatic or aromatic acyl, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, or a substituted or unsubstituted heterocyclic group, a carbalkoxy, a carbaryloxy, —SO₃H and —SO₃R³, —P(O)(OH)₂, and P(O)(OR³)₂.

6. The method of claim 4, wherein the compound is

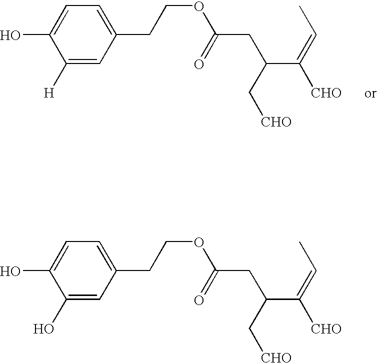

7. The method according to claim 4; wherein the one or more isolated and purified compounds is administered in combination with one or more active ingredients which have favorable effects on metabolic disturbances or disorders.

8. The method according to claim 4, wherein the one or more isolated and purified compounds is administered in combination with a therapeutically effective amount of one or more anti-diabetic drugs.

9. The method according to claim 4, wherein the one or more isolated and purified compounds is administered in combination with a therapeutically effective amount of one or more lipid modulators.

10. A method of treating or preventing one or more symptoms associated with disorders of fatty acid metabolism or glucose utilization disorders in a patient in need thereof comprising administering to said patient a therapeutically effective amount of the one or more isolated and purified compound of formulae (I) and (II) 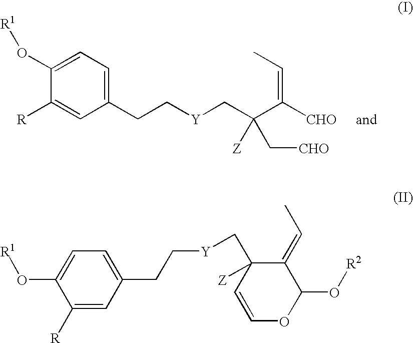
wherein R is hydrogen, hydroxyl or —OR¹;
wherein R¹ is hydrogen or a chemical moiety that can be cleaved in vivo to release a hydroxyl group;
wherein R² is hydrogen or a hydroxyl protecting group, —SO₃H and —SO₃R³ where R³ is a saturated or unsaturated aliphatic group; a substituted or unsubstituted saturated or unsaturated alicyclic group;
wherein R³ is a saturated or unsaturated aliphatic group, substituted or unsubstituted aliphatic group, substituted or unsubstituted saturated or unsaturated alicyclic group, substituted or unsubstituted aromatic group, substituted or unsubstituted heteroaromatic group, substituted or unsubstituted heterocyclic group, glucuronide or glucuronide ester, —P(O)(OH)₂, and —P(O)(OR³) 2;
wherein Y is —(CH₂)ₘ— where m=1 or 2, —OC(O)—, —O(CH₂)ₘ— where m=1 or 2, or —S(O)ₙ(CH₂)ₘ— where m=1 or 2; n=0, 1 or 2; and,
wherein Z is hydrogen, CH₃, F, Cl, Br or I.

11. The method of claim 10 wherein R¹ is aliphatic or aromatic acyl, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, or a substituted or unsubstituted heterocyclic group, a carbalkoxy, a carbaryloxy, —SO₃H and —SO₃R³, —P(O)(OH)₂, or —P(O)(OR³)₂.

12. The method of claim 10, wherein the compound is

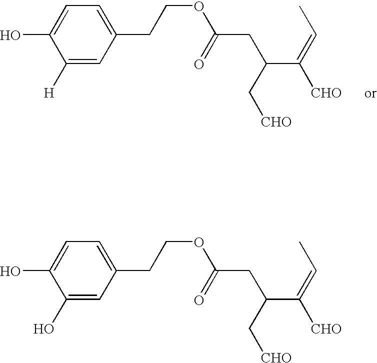

13. The method according to claim 10, wherein the one or more isolated and purified compounds is administered in combination with at least one further active ingredient for the treatment or prevention of disorders of fatty acid metabolism and glucose utilization disorders.

14. A method of treating or preventing disorders involving insulin resistance comprising administering to a patient in need thereof a therapeutically effective amount of the one or more isolated and purified compounds of formulae (I) and (II) 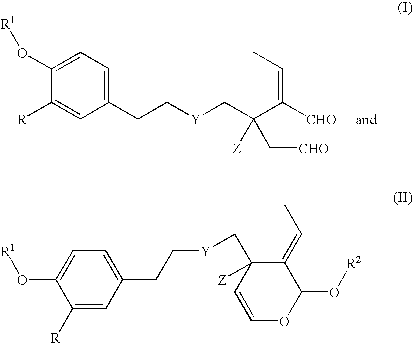
wherein R is hydrogen, hydroxyl or —OR¹;
wherein R¹ is hydrogen or a chemical moiety that can be cleaved in vivo to release a hydroxyl group;
wherein R² is hydrogen or a hydroxyl protecting group, —SO₃H and —SO₃R³ where R³ is a saturated or unsaturated aliphatic group; a substituted or unsubstituted saturated or unsaturated alicyclic group;
wherein R³ is a saturated or unsaturated aliphatic group, substituted or unsubtituted aliphatic group, substituted or unsubstituted saturated or unsaturated alicyclic group, substituted or unsubstituted aromatic group, substituted or unsubstituted heteroaromatic group, substituted or unsubstituted heterocyclic group, glucuronide or glucuronide ester, —P(O)(OH)₂, and —P(O)(OR³)₂;
wherein Y is —(CH₂)ₘ— where m=1 or 2, —OC(O)—, —O(CH₂)ₘ— where m=1 or 2, or —S(O)ₙ(CH₂)ₘ— where m=1 or 2; n=0, 1 or 2; and,
wherein Z is hydrogen, CH₃, F, Cl, Br or I.

15. The method of claim 14 wherein R¹ is aliphatic or aromatic acyl, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, or a substituted or unsubstituted heterocyclic group, a carbalkoxy, a carbaryloxy, —SO₃H and —SO₃R³, —P(O)(OH)₂, or —P(O)(OR³)₂.

16. The method of claim 14, wherein the compound is

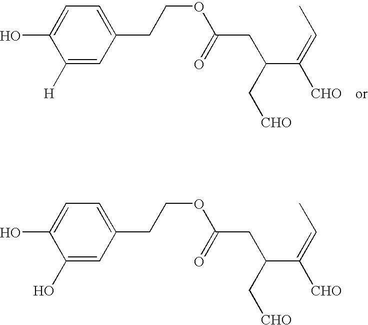

17. The method according to claim 14, wherein the disorder involving insulin resistance is diabetes mellitus.

18. The method according to claim 14, wherein the one or more isolated and purified compounds is administered in combination with at least one further active ingredient for the treatment and/or prevention of disorders in which insulin resistance is involved.

19. A method of treating or preventing dyslipidemias and their complications comprising administering to a patient in need thereof a therapeutically effective amount of the one or more isolated and purified compounds of formulae (I) and (II) 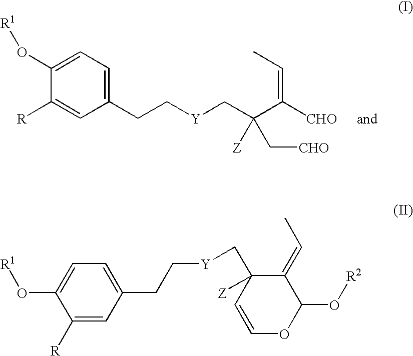
wherein R is hydrogen, hydroxyl or —OR¹;
wherein R¹ is hydrogen or a chemical moiety that can be cleaved in vivo to release a hydroxyl group;
wherein R² is hydrogen or a hydroxyl protecting group, —SO₃H and —SO₃R³ where R³ is a saturated or unsaturated aliphatic group; a substituted or unsubstituted saturated or unsaturated alicyclic group;
wherein R³ is a saturated or unsaturated aliphatic group, substituted or unsubtituted aliphatic group, substituted or unsubstituted saturated or unsaturated alicyclic group, substituted or unsubstituted aromatic group, substituted or unsubstituted heteroaromatic group, substituted or unsubstituted heterocyclic group, glucuronide or glucuronide ester, —P(O)(OH)₂, and —P(O)(OR³)₂;
wherein Y is —(CH₂)ₘ— where m=1 or 2, —OC(O)—, —O(CH₂)ₘ— where m=1 or 2, or —S(O)ₙ(CH₂)ₘ— where m=1 or 2; n=0, 1 or 2; and,
wherein Z is hydrogen, CH₃, F, Cl, Br or I.

20. The method of claim 19, wherein R¹ is aliphatic or aromatic acyl, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, or a substituted or unsubstituted heterocyclic group, a carbalkoxy, a carbaryloxy, —SO₃H and —SO₃R³, —P(O)(OH)₂, or —P(O)(OR³)₂.

21. The method of claim 19, wherein the compound is

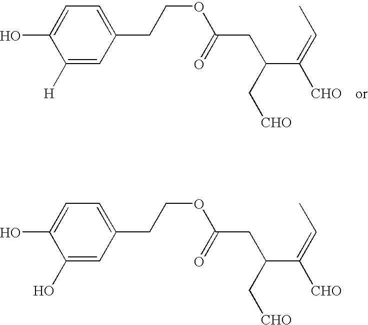

22. A method of treating or preventing conditions associated with metabolic syndrome X comprising administering to a patient in need thereof a therapeutically effective amount of the one or more isolated and purified compounds of formulae (I) and (II) 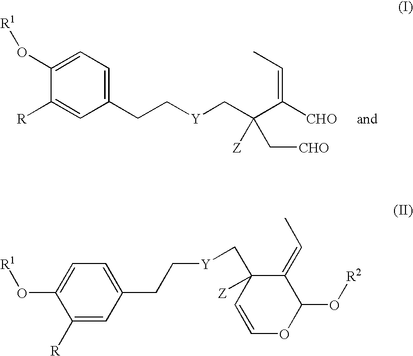
wherein R is hydrogen, hydroxyl or —OR¹;
wherein R¹ is hydrogen or a chemical moiety that can be cleaved in vivo to release a hydroxyl group;
wherein R² is hydrogen or a hydroxyl protecting group, —SO₃H and —SO₃R³ where R³ is a saturated or unsaturated aliphatic group; a substituted or unsubstituted saturated or unsaturated alicyclic group;
wherein R³ is a saturated or unsaturated aliphatic group, substituted or unsubtituted aliphatic group, substituted or unsubstituted saturated or unsaturated alicyclic group, substituted or unsubstituted aromatic group, substituted or unsubstituted heteroaromatic group, substituted or unsubstituted heterocyclic group, glucuronide or glucuronide ester, —P(O)(OH)₂, and —P(O)(OR³)₂;
wherein Y is —(CH₂)ₘ— where m=1 or 2, —OC(O)—, —O(CH₂)ₘ— where m=1 or 2, or —S(O)ₙ(CH₂)ₘ— where m=1 or 2; n=0, 1 or 2; and,
wherein Z is hydrogen, CH₃, F, Cl, Br or I.

23. The method of claim 22 wherein R¹ is aliphatic or aromatic acyl, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, or a substituted or unsubstituted heterocyclic group, a carbalkoxy, a carbaryloxy, —SO₃H and —SO₃R³, —P(O)(OH)₂, or —P(O)(OR³)₂.

24. The method of claim 22, wherein the compound is [chemical structure]
